(19) United States
(12) Patent Application Publication
(10) Pub. No.: US 2007/0155707 A1
(43) Pub. Date: Jul. 5, 2007

(54) IONIZABLE INHIBITORS OF FATTY ACID AMIDE HYDROLASE

(75) Inventors: Olivier Dasse, Foothill Ranch, CA (US); David Putman, Irvine, CA (US); Timothy R. Compton, San Clemente, CA (US)

Correspondence Address: WILSON SONSINI GOODRICH & ROSATI, 650 PAGE MILL ROAD, PALO ALTO, CA, 94304-1050, US

(73) Assignee: Kadmus Pharmaceuticals, Inc., Irvine, CA (US)

(21) Appl. No.: 11/561,774

(22) Filed: Nov. 20, 2006

Related U.S. Application Data
(60) Provisional application No. 60/755,035, filed on Dec. 29, 2005.
(60) Provisional application No. 60/828,753, filed on Oct. 9, 2006.

(51) Int. Cl. A61K 31/41; A61K 31/325; A61K 31/66

(52) U.S. Cl. 514/114; 514/381; 514/489; 548/253; 560/132

(57) ABSTRACT

Pharmacological inhibition of fatty acid amide hydrolase (FAAH) activity leads to increased levels of fatty acid amides. Esters of alkylcarbamic acids are disclosed that are inhibitors of FAAH activity. Compounds disclosed herein inhibit FAAH activity. Described herein are processes for the preparation of esters of alkylcarbamic acid compounds, compositions that include them, and methods of use thereof.

### RELATED APPLICATIONS

This application claims benefit of U.S. Provisional Application No. 60/755,035 entitled “Derivatives of Carbamates, Methods of their Preparation, Inhibition of Fatty Acid Amide Hydrolase, and Therapuetic Uses Thereof” filed Dec. 29, 2005; and U.S. Provisional Application No. 60/828,753 entitled “Ionizable Inhibitors of Fatty Acid Amide Hydrolase” filed Oct. 9, 2006, both of which are herein incorporated by reference.

### FIELD OF THE INVENTION

Described herein are compounds, methods of making such compounds, pharmaceutical compositions and medicaments containing such compounds, and methods of using such compounds and compositions to inhibit the activity of fatty acid amide hydrolase (FAAH).

### BACKGROUND OF THE INVENTION

Fatty acid amide hydrolase (FAAH) is an enzyme that hydrolyzes the fatty acid amide (FAA) family of endogenous signaling lipids. General classes of FAAs include the N-acylethanolamines (NAEs) and fatty acid primary amides (FAPAs). Examples of NAEs include anandamide (AEA), palmitoylethanolamide (PEA) and oleoylethanolamide (OEA). Pharmacological inhibition of FAAH activity results in increases in the levels of these fatty acid amides.

### SUMMARY OF THE INVENTION

Compounds, compositions and methods for inhibiting the activity of fatty acid amide hydrolase (FAAH) are provided. Among the compounds provided herein are compounds that are inhibitors of fatty acid amide hydrolase (FAAH). Processes for the preparation of compounds that inhibit the activity of fatty acid amide hydrolase, compositions that include the compounds, as well as methods of use thereof are provided.

Compounds provided herein include those that have a structure of Formula (II) and pharmaceutically acceptable salts, solvates, esters, acids and prodrugs thereof. In certain embodiments, isomers and chemically protected forms of compounds having a structure represented by Formula (II) are also provided.

Formula (II) is as follows:

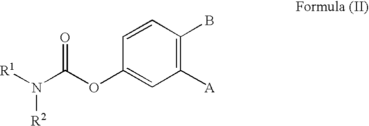

wherein:
R¹ is an optionally substituted group selected from among C₃-C₉ cycloalkyl and —C₁-C₄alkyl(C₃-C₉cycloalkyl);
R² is H or an optionally substituted alkyl;
if R¹ is an optionally substituted C₃-C₉cycloalkyl or an optionally substituted —C₁-C₄alkyl(C₃-C₉ cycloalkyl), then any carbon of the R¹ carbocyclic ring can be optionally substituted by both Y and Z;
Y and Z are independently selected from among halogen or methyl; or
Y and Z taken together form an oxo (═O);
one of A Or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, NR⁹C(O)N(R⁹)—(CH₂)ₙ—, —S(O)—(CH₂)ₙ—, —S(O)₂—(CH₂)ₙ—, —S(O)₂NH—(CH₂)ₙ—, —C(═NR¹⁰)N(R⁹)—(CH₂)ₙ—, and —NR⁹C(═NR¹⁰)N(R)—(CH₂)ₙ—;
G is tetrazolyl, —NHS(═O)₂R⁸, —S(═O)₂NHR⁹, —OH, —SH, —N(R⁹)₂, —C(O)NHC(O)R⁸, —C(O)NHS(═O)₂R⁸, —S(═O)₂NHC(O)R⁸, —S(═O)₂NHC(O)NHR⁸, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, carboxylic acid bioisosteres, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 0, 1, or 2; and
pharmaceutically acceptable salts, pharmaceutically acceptable N-oxides, pharmaceutically active metabolites, pharmaceutically acceptable prodrugs, or pharmaceutically acceptable solvates thereof.

For any and all of the embodiments, substituents can be selected from among from a subset of the listed alternatives. For example, in some embodiments, one of A or B is -L-G and the other is H or an optionally substituted C₁-C₆ alkyl. In other embodiments, one of A or B is -L-G and the other is H. In some embodiments, A is -L-G. In some other embodiments, B is -L-G.

In certain embodiments, R² is H.

In some embodiments, L is a bond, or an optionally substituted group selected from among C₁-C₆ alkylene, C₁-C₆ketoalkylene, —C(O)NR⁹—(CH₂)ₙ—, —NR⁹—C(O)—(CH₂)ₙ—, —S(O)₂—(CH₂)ₙ—, and —S(O)₂NH—(CH₂)ₙ—. In some embodiments, L is a bond, or an optionally substituted group selected from among C₁-C₆ alkylene, and C₁-C₆ ketoalkylene. In other embodiments, L is a bond.

In certain embodiments, if L is not a bond, L taken together with A or B can form a carbocyclic ring.

In some embodiments, G is —N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, or —C(O)NR⁹C(═CHR¹⁰)N(R⁹)₂.

In some embodiments, G is —N(R⁹)₂, —C(═NR¹⁰)N(R⁹)₂, or —NR⁹C(═NR¹⁰)N(R⁹)₂. In some other embodiments, each R¹⁰ is H.

In some embodiments, G is tetrazolyl, —NHS(═O)₂R⁸, —S(═O)₂NHR⁹, —OH, —SH, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)ₙOH; —OS(O)ₙOH; or —NR⁹S(O)_{y}OH. In other embodiments, G is tetrazolyl, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₙOH, —OP(═O)OR⁸OH, —OP(═O)R⁸OH, —P(═O)OR⁸OH; —P(═O)R⁸OH, or —S(O)_{y}OH. In some other embodiments, G is tetrazolyl, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₓOH, or —S(O)_{y}OH; x is 1; and y is 1 or 2. In yet some other embodiments, G is tetrazolyl, —CO₂H, or a carboxylic acid bioisostere.

In other embodiments, R¹ is an optionally substituted C₃-C₉cycloalkyl. In yet other embodiments, R¹ is an optionally substituted —C₁-C₄alkyl-(C₃-C₉cycloalkyl). In some embodiments, R¹ is selected from among cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl. In other embodiments, R¹ is selected from among cyclohexyl and cyclohexylmethyl.

In some embodiments, R¹ is selected from among n-butyl, sec-butyl, iso-butyl, tert-butyl, pentyl, neopentyl, hexyl, cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl. In certain embodiments, R¹ is selected from among sec-butyl, iso-butyl, tert-butyl, cyclopropyl, cyclobutyl, cyclopentyl, cyclohexyl, cycloheptyl, cyclopropylmethyl, cyclobutylmethyl, cyclopentylmethyl, cyclohexylmethyl and cycloheptylmethyl. In some of the embodiments provided herein, R¹ is selected from among sec-butyl, iso-butyl, tert-butyl, cyclopropyl, cyclopentyl, cyclohexyl, cyclopropylmethyl, cyclopentylmethyl, and cyclohexylmethyl. In some embodiments, R¹ is selected from among cyclohexyl, cyclopropylmethyl and cyclohexylmethyl. In certain embodiments, R¹ is selected from among cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl. In other embodiments, R¹ is selected from among cyclohexyl and cyclohexylmethyl.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In one embodiment, provided herein is a compound selected from among: 3-(cyclohexylcarbamoyloxy)benzoic acid (Compound 1); 3-(cyclohexylmethylcarbamoyloxy)benzoic acid (Compound 2); 4-(cyclohexylcarbamoyloxy)benzoic acid (Compound 3); 4-(cyclohexylmethylcarbamoyloxy)benzoic acid (Compound 4); 2-(3-((cyclohexylmethyl)carbamoyl)phenyl)acetic acid (Compound 7); 5-(3-(cyclohexylcarbamoyl)phenyl)-5-oxopentanoic acid (Compound 8); 5-(3-((cyclohexylmethyl)carbamoyl)phenyl)-5-oxopentanoic acid (Compound 9); 3-(1H-tetrazol-5-yl)phenyl cyclohexylcarbamate (Compound 10); 3-(1H-tetrazol-5-yl)phenyl cyclohexylmethylcarbamate (Compound 11); 4-(3-(cyclohexylcarbamoyl)phenyl)-4-oxobutanoic acid (Compound 12); and 4-(3-((cyclohexylmethyl)carbamoyl)phenyl)-4-oxobutanoic acid (Compound 13).

In some embodiments, provided herein is a compound having the structure of Formula (I):

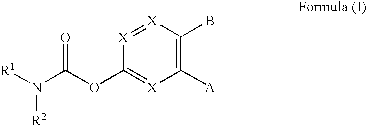

wherein:
R¹ is an optionally substituted group selected from among C₁-C₆ alkyl, C₃-C₉ cycloalkyl and —C₁-C₄alkyl(C₃-C₉cycloalkyl);
R² is H or an optionally substituted alkyl;
if R¹ is an optionally substituted C₃-C₉ cycloalkyl or an optionally substituted —C₁-C₄alkyl(C₃-C₉cycloalkyl), then any carbon of the R¹ carbocyclic ring can be optionally substituted by both Y and Z;
Y and Z are independently selected from among halogen or methyl; or
Y and Z taken together form an oxo (═O);
one of A or B is -L-G and the other is selected from among H and an optionally substituted C₁-C₆ alkyl; or
A and B are each independently selected from among H and an optionally substituted C₁-C₆ alkyl provided that at least one X is N;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —S(O)₂NH—(CH₂)ₙ—, —C(═NR¹⁰)N(R⁹)—(CH₂)ₙ—, and —NR⁹C(═NR¹⁰)N(R⁹)—(CH₂)ₙ—;
G is tetrazolyl, —NHS(═O)₂R⁸, —S(═O)₂NHR⁹, —OH, —SH, —N(R⁹)₂, —C(O)NHC(O)R⁸, —C(O)NHS(═O)₂R⁸, —S(═O)₂NHC(O)R⁸, —S(═O)₂NHC(O)NHR⁸, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, carboxylic acid bioisosteres, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; or —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 0, 1, or 2; and
   pharmaceutically acceptable salts, pharmaceutically acceptable N-oxides, pharmaceutically active metabolites, pharmaceutically acceptable prodrugs, or pharmaceutically acceptable solvates thereof.

In a further aspect are provided pharmaceutical compositions, which include a therapeutically effective amount of at least one of any of the compounds herein, or a pharmaceutically acceptable salt, pharmaceutically acceptable N-oxide, pharmaceutically active metabolite, pharmaceutically acceptable prodrug, or pharmaceutically acceptable solvate. In certain embodiments, the compositions provided herein further include a pharmaceutically acceptable diluent, excipient and/or binder.

Pharmaceutical compositions formulated for administration by an appropriate route and means containing effective concentrations of one or more of the compounds provided herein, or pharmaceutically effective derivatives thereof, that deliver amounts effective for the treatment, prevention, or amelioration of one or more symptoms of diseases, disorders or conditions that are modulated or otherwise affected by FAAH activity, or in which FAAH activity is implicated, are provided. The effective amounts and concentrations are effective for ameliorating any of the symptoms of any of the diseases, disorders or conditions disclosed herein.

In certain embodiments, provided herein is a pharmaceutical composition containing: i) a physiologically acceptable carrier, diluent, and/or excipient; and ii) one or more compounds provided herein.

In one aspect, provided herein are methods for treating a patient by administering a compound provided herein. In some embodiments, provided herein is a method of inhibiting the activity of fatty acid amide hydrolase or of treating a disease, disorder, or condition, which would benefit from inhibition of fatty acid amide hydrolase activity in a patient, which includes administering to the patient a therapeutically effective amount of at least one of any of the compounds herein, or pharmaceutically acceptable salt, pharmaceutically acceptable N-oxide, pharmaceutically active metabolite, pharmaceutically acceptable prodrug, or pharmaceutically acceptable solvate.

In certain embodiments, compounds and compositions provided herein are effective for the treatment, prevention, or amelioration of one or more symptoms of diseases, disorders or conditions that are selected from among acute or chronic pain, eating disorders, cardiovascular diseases, metabolic diseases, disorders or conditions, renal ischemia, cancers, disorders of the immune system, allergic diseases, parasitic, viral or bacterial infectious diseases, inflammatory diseases, osteoporosis, ocular conditions, pulmonary conditions, gastrointestinal diseases and urinary incontinence.

In other embodiments, compounds provided herein are effective for the treatment, prevention, or amelioration of one or more symptoms of diseases, disorders or conditions that are selected from among pain, nociceptive pain, neuropathic pain, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, Parkinson's disease, muscle spasticity, epilepsy, obesity, hyperlipidemia, insulin resistance syndrome, fatty liver disease, obesity, atherosclerosis, arteriosclerosis, metabolic disorders, feeding and fasting, alteration of appetite, hypertension, septic shock, cardiogenic shock, intestinal inflammation and motility, irritable bowel syndrome, colitis, diarrhea, ileitis, ischemia, cerebral ischemia, hepatic ischemia, myocardial infarction, arthritis, rheumatoid arthritis, spondylitis, shoulder tendonitis or bursitis, gouty arthritis, aolymyalgia rheumratica, thyroiditis, hepatitis, inflammatory bowel diseases, asthma, multiple sclerosis, chronic obstructive pulmonary disease (COPD), allergic rhinitis, and cardiovascular diseases.

Compounds provided herein are irreversible inhibitors of fatty acid amide hydrolase (FAAH). Compounds provided herein increase the levels of some endogenous fatty acid amides. Compounds provided herein increase the levels of endogenous fatty acid amides selected from among AEA, OEA and PEA.

In some embodiments, compounds provided herein do not substantially cross the blood-brain barrier.

Fatty acid amide hydrolase (FAAH) is present throughout the body. In some cases, it is preferable to restrict FAAH inhibitors to peripheral tissues so as to minimize or eliminate any psychotropic effects. In some embodiments, compounds provided herein preferentially inhibit FAAH activity in peripheral tissues and fluids and minimize potentially undesired central nervous system side effects. In some embodiments, the incorporation of an ionizable group into a FAAH inhibitor compound decreases the ability of the compound to cross the blood-brain-barrier. In some embodiments, incorporation of an ionizable group into a FAAH inhibitor provides a FAAH inhibitor compound that preferentially inhibits FAAH activity in peripheral tissues. In some embodiments, the incorporation of an ionizable group into a FAAH inhibitor compound, such as, for example, compounds disclosed herein, may be used to inhibit FAAH activity in peripheral tissues in preference to CNS tissues. In some embodiments, the incorporation of an ionizable group into a FAAH inhibitor compound provides a FAAH inhibitor compound that does not substantially cross the blood-brain-barrier and is not an effective therapuetic in neural disorders.

In some embodiments, compounds provided herein are FAAH inhibitor compounds that are ionizable at physiological pH and do not substantially cross the blood brain barrier. Compounds that are ionizable at physiological pH are charged and do not substantially cross the blood brain barrier.

In some embodiments, compounds provided herein, after administration to a mammal, result in plasma AUC values that are at least 5 times greater than brain tissue AUC values, provided that the administration is conducted as described in Example 15. In other embodiments, compounds provided herein, after administration to a mammal, result in plasma AUC values that are at least 5 times, at least 6 times, at least 8 times, at least 10 times, at least 12 times, at least 14 times, at least 16 times, at least 18 times, at least 20 times, or at least 30 times greater than the brain tissue AUC values.

In some embodiments, compounds provided herein are administered to a human.

In some embodiments, compounds provided herein are orally administered.

In some embodiments, compounds provided herein are used for inhibiting fatty acid amide hydrolase (FAAH) activity. In some embodiments, compounds provided herein are used for inhibiting the activity of fatty acid amide hydrolase activity or for the treatment of a disease or condition that would benefit from inhibition of fatty acid amide hydrolase activity.

In other embodiments, compounds provided herein are used for the formulation of a medicament for the inhibition of fatty acid amide hydrolase (FAAH).

In certain embodiments, compounds and compositions provided herein are effective for the treatment, prevention, or amelioration of one or more symptoms of diseases, disorders or conditions that are selected from among acute or chronic pain, dizziness, vomiting, nausea, eating disorders, neurological and psychiatric pathologies, acute or chronic neurodegenerative diseases, epilepsy, sleep disorders, cardiovascular diseases, renal ischemia, cancers, disorders of the immune system, allergic diseases, parasitic, viral or bacterial infectious diseases, inflammatory diseases, osteoporosis, ocular conditions, pulmonary conditions, gastrointestinal diseases and urinary incontinence.

In some embodiments, compounds and compositions provided herein are effective for the treatment, prevention, or amelioration of one or more symptoms of diseases, disorders or conditions that are selected from among pain, nociceptive pain, neuropathic pain, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, depression, anxiety, generalized anxiety disorder (GAD), obsessive compulsive disorders, stress, stress urinary incontinence, attention deficit hyperactivity disorders, schizophrenia, psychosis, Parkinson's disease, muscle spasticity, epilepsy, obesity, hyperlipidemia, insulin resistance syndrome, fatty liver disease, obesity, atherosclerosis, arteriosclerosis, metabolic disorders, feeding and fasting, alteration of appetite, memory, aging, hypertension, septic shock, cardiogenic shock, intestinal inflammation and motility, irritable bowel syndrome, colitis, diarrhea, ileitis, ischemia, cerebral ischemia, hepatic ischemia, myocardial infarction, cerebral excitotoxicity, seizures, febrile seizures, neurotoxicity, neuropathies, sleep, induction of sleep, prolongation of sleep, insomnia, arthritis, rheumatoid arthritis, spondylitis, shoulder tendonitis or bursitis, gouty arthritis, aolymyalgia rheumatica, thyroiditis, hepatitis, inflammatory bowel diseases, asthma, multiple sclerosis, chronic obstructive pulmonary disease (COPD), allergic rhinitis, and cardiovascular diseases.

In certain other embodiments, compounds and compositions provided herein are effective for the treatment, prevention, or amelioration of one or more symptoms of pain and/or inflammation.

In one aspect, provided herein is a method of inhibiting fatty acid amide hydrolase activity in a mammal, which includes administering to said mammal a therapeutically effective amount of a compound or composition provided herein. In some embodiments the mammal is a human. In other embodiments, compound or composition is orally administered.

In another aspect, a compound provided herein is used for the formulation of a medicament for the inhibition of fatty acid amide hydrolase (FAAH).

Articles of manufacture containing packaging material, a compound or composition or pharmaceutically acceptable derivative thereof provided herein, which is effective for inhibiting the activity of fatty acid amide hydrolase (FAAH), within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for inhibiting the activity of fatty acid amide hydrolase (FAAH), are provided.

Any of the combinations of the groups described above for the various variables is contemplated herein.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. All references cited herein, including patents, patent applications, and publications, are hereby incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The novel features are set forth with particularity in the appended claims. A better understanding of the features and advantages of the present disclosure will be obtained by reference to the following detailed description that sets forth illustrative embodiments, in which the principles described herein are utilized.

Disclosed herein are compounds that inhibit the activity the activity of fatty acid amide hydrolase (FAAH), compositions that include the compounds, and methods of their use. Compounds disclosed herein are inhibitors of fatty acid amide hydrolase (FAAH) and are useful in the treatment of diseases, disorders, or conditions that would benefit from the inhibition of fatty acid amide hydrolase and increases in endogenous fatty acid amides.

### The Endocannabinoid System

The endocannabinoid signaling system is composed of three elements (Lambert et al. J. Med. Chem. 2005, vol. 48, no. 16, 5059-5087). The first is represented by the G protein-coupled receptors that bind endogenous and exogenous cannabinoid ligands. Two such receptors have been identified, the CB₁ receptor, which is found almost everywhere in the body, but is most abundant in the central nervous system (CNS) (Freund et al. Physiol. Rev. 2003; 83:1017-1066); and the CB₂ receptor, which is primarily expressed in immune cells and in hematopoietic cells, but is also present at low levels in the brain (Munro et al. Nature, 1993; 365:61-65; Van Sickle et al. Science 2005; 310:329-332; Hanus et al., Proc. Nat. Acad. Sci., U.S.A., 1999; 96:14228-14233).

The second element is represented by the endocannabinoids, naturally occurring lipid molecules that bind to and activate cannabinoid receptors (Devane et al. Science 1992; 258: 1946-1949; Mechoulam et al. Biochem. Pharmacol. 1995; 50:83-90; Sugria et al. Biochem. Biophys. Res. Commun. 1995; 215:89-97), are generated on demand by nuerons and other cells (Di Marzo et al. Nature 1994; 372:686-691; Giuffrida et al. Nat. Nuerosci. 1999; 2:358-363; Stella et al. Nature 2001; 388:773-778), and are rapidly eliminated (Beltramo et al. FEBS Lett. 1997; 403:263-267; Stella et al. Nature 2001; 388:773-778).

The third element is represented by the proteins involved in the formation and elimination of the various endocannabinoid ligands (Piomelli, D. Nat. Rev. Neurosci. 2003; 4:873-884).

Cannabinoid receptors can be activated by endocannabinoids, as well as synthetic ligands.

Anandamide (arachidonoylethanolamide) was the first endocannabinoid substance to be discovered (Devane et al. Science 1992; 258:1946-1949; Piomelli, D. Nat. Rev. Neurosci. 2003; 4:873-884). Current evidence indicates that this lipid-derived mediator is released upon demand by stimulated nuerons (Di Marzo et al. Nature, 1994; 372:686-691; Giuffrida et al. Nat. Neurosci. 1999, 2:358-363); activates cannabinoid receptors with high potency (Devane et al. Science 1992; 258:1946-1949), and is rapidly eliminated through a two-step process consisting of carrier-mediated internalization followed by intracellular hydrolysis (metabolism) (Beltramo et al. Science 1997; 277:1094-1097; Di Marzo et al. Nature 1994; 372:686-691; Hillard et al. J. Lipid Res. 1997; 38:2383-2398).

The endocannabinoids anandamide and 2-arachidonylglycerol (2-AG), both of which produce most of their effects by binding to the CB₁ receptor, have been shown to be tonically released and can control basal nociceptive thresholds (Meng et al., Nature 1998; Sep. 24; 395(6700):381-3). In particular, anandamide acts as a CB₁ agonist and exhibits pharmacological activity in mice comparable to other synthetic cannabinoids.

### Fatty Acid Amide Hydrolase (FAAH)

Fatty acid amide hydrolase (FAAH) is an enzyme that hydrolyzes the fatty acid amide (FAA) family of endogenous signaling lipids. General classes of fatty acid amides include the N-acylethanolamines (NAEs) and fatty acid primary amides (FAPAs). Examples of NAEs include anandamide (AEA), palmitoylethanolamide (PEA) and oleoylethanolamide (OEA). An example of FAPAs includes 9-Z-octadecenamide or oleamide. (McKinney MK, Cravatt BF. 2005. Annu Rev Biochem 74:411-32)]. FAAH can act as a hydrolytic enzyme not only for fatty acid ethanolamides and primary amides, but also for esters, such as, for example, 2-arachidonylglycerol (2-AG) (Mechoulam et al. Biochem. Pharmacol. 1995; 50:83-90; Stella et al. Nature, 1997; 388:773-778; Suguria et al. Biochem. Biophys. Res. Commun. 1995; 215:89-97)

FAAH is abundantly expressed throughout the CNS (Freund et al. Physiol. Rev. 2003; 83:1017-1066) as well as in peripheral tissues, such as, for example, in the pancreas, brain, kidney, skeletal muscle, placenta, and liver (Giang, D. K. et al. Molecular Characterization of Human and Mouse Fatty Acid Amide Hydrolases. Proc. Natl. Acad. Sci. U.S.A. 1997, 94, 2238-2242; Cravatt et al. PNAS, 2004, vol. 101, no. 29, 10821-10826.).

Anandamide, or arachidonylethanolamide, is a NAE that acts as an endogenous ligand for the cannabinoid type 1 (CB₁) receptor (Devane W A, et al. 1992. Science 25 8:1946-49). Anandamide is rapidly eliminated through a two-step process consisting of carrier-mediated transport followed by intracellular hydrolysis by FAAH. The hydrolysis of anandamide by FAAH results in the formation of arachidonic acid and ethanolamine. The current postulated catalytic mechanism for hydrolyisis of anandamide by FAAH involves nucleophilic attack of amino acid residue Serine 241 of FAAH on the amide moiety of anandamide, resulting in the formation of arachidonic acid and ethanolamine (Deutsch et al. The fatty acid amide hydrolase (FAAH) Prostaglandins, Leukotrienes and Essential Fatty Acids (2002) 66 (2&3), 201-210; Alexander et al. Chemistry & Biology, vol. 12, 1179-1187; 2005.).

Mutant mice lacking the gene encoding for FAAH display a profound reduction in hydrolysis activity for anandamide and other fatty acid amides and show signs of enhanced anandamide activity at cannabinoid receptors, leading to observable physiological phenomena such as reduced pain sensation (Cravatt B F, et al. 2001. Proc Nat Acad Sci USA 98: 9371-9376). This suggests that therapeutic agents that alter the activity of the FAAH enzyme can increase the actions of anandamide and other fatty acid amides in the body. Such agents may also avoid the multiple, often undesirable effects produced by indiscriminant activation of cannabinoid receptors by administration of Δ9-THC (the active ingredient in marijuana) and other direct-acting cannabinoids.

Many endogenous fatty acid amides, other than anandamide, do not bind the CB₁ receptor. Several of these lipids have been shown to produce specific cellular and behavioral effects, and may represent a large family of endogenous signalling lipids that act in vivo on receptor systems distinct from CB₁. These include palmitoylethanolamide (PEA) (Calignano A, et al. 1998. Nature 394:277-81; Jaggar S I, et al. 1998. Pain 76:189-99; Franklin A, Parmentier-Batteur et al. 2003. J Neurosci 23: 7767-75), stearoylethanolamide (SEA) (Terrazino et al. 2004 FASEB J: 18:1580-82; Maccarrone M, et al. 2002. Biochem J 366:137-44), and oleoylethanolamide (OEA) (deFonseca F R, et al. 2001. Nature 414:209-12; Fu J, et al. 2003. Nature 425:90-93; Fu J, et al. 2005. Neuropharmacology 48(8): 1147-53). Both OEA and PEA have been shown to activate peroxisome proliferator-activated receptor alpha (PPAR-alpha) (Fu J, et al. 2003. Nature 425:90-93; Guzman M, et al. 2004, J Biol Chem 279(27): 27849-54; Lo Verme J, et al. 2005. Cell Mol Life Sci 62(6): 708-16; Lo Verme J, et al 2005. Life Sci 77(14): 1685-98; Lo Verme J, et al. 2005. Mol Pharmacol 67(1): 15-9). Through these actions, OEA and PEA can regulate several biological pathways including, but not limited to, feeding, metabolism, pain and inflammation. Therefore, agents that alter FAAH enzymatic activity can regulate the levels of a variety of fatty acid amides in vivo that, in turn, have therapeutic actions through a variety of targets.

Without being bound by theory, it is thought that certain fatty acid amides, such as, for example, OEA, act through the peroxisome proliferator-activated receptor α (PPAR-α) to regulate diverse physiological processes, including, e.g., feeding and lipolysis. Consistent with this, human adipose tissue has been shown to bind and metabolize endobannabinoids such as anandamide and 2-arachidonylglycerol. See Spoto et al., Aug. 22, 2006, Biochimie (E-publication ahead of print); and Matias et al. (2006), J. Clin. Endocrin. & Met., 91(8):3171-3180. Thus, inhibiting FAAH activity in vivo leads to reduced body fat, body weight, caloric intake, and liver triglyceride levels. However, unlike, other anti-lipidemic agents that act through PPAR-; e.g., fibrates, FAAH inhibitors do not cause adverse side effects such as rash, fatigue, headache, erectile dysfunction, and, more rarely, anemia, leukopenia, angioedema, and hepatitis. See, e.g., Muscari et al. (2002), Cardiology, 97:115-121. An additional therapeutic property of FAAH inhibitors is that due to their ability to elevate anandamide levels, they effectively alleviate depression and anxiety, conditions often associated with energy metabolism disorders (EMDs) such as obesity. See Simon et al. (2006), Archives of Gen. Psychiatry, 63(7):824-830. In some embodiments, FAAH inhibitor compounds may be peripherally restricted and may not substantially affect neural disorders, such as, for example, depression and anxiety. Finally, agonism of cannabinoid receptors has also been shown to reduce the progression of atherosclerosis in animal models. See Steffens et al. (2005), Nature, 434:782-786; and Steffens et al. (2006), Curr. Opin. Lipid., 17:519-526. Thus, increasing the level of endogenous cannabinergic fatty acid amides (e.g., anandamide) is expected to effectively treat or reduce the risk of developing atherosclerosis.

Many fatty acid amides are produced on demand and rapidly degraded by FAAH. As a result, hydrolysis by FAAH is considered to be one of the essential steps in the regulation of fatty acid amide levels in the central nervous system as well as in peripheral tissues and fluids. The broad distribution of FAAH combined with the broad array of biological effects of fatty acid amides (both endocannabinoid and non-endocannabinoid mechanisms) suggests that inhibition of FAAH may lead to altered levels of fatty acid amides in many tissues and fluids and may be useful to treat many different conditions. FAAH inhibitors increase the levels of endogenous fatty acid amides. FAAH inhibitors block the degradation of endocannabinoids and increase the tissue levels of these endogenous substances. FAAH inhibitors can be used in this respect in the prevention and treatment of pathologies in which endogenous cannabinoids and or any other substrates metabolized by the FAAH enzyme are involved.

Inhibition of FAAH is expected to lead to an increase in the level of anadamide and other fatty acid amides. This increase in fatty acid amides may lead to an increase in the noiceptive threshold. Thus, in one embodiment, inhibitors of FAAH are useful in the treatment of pain. Such inhibitors might also be useful in the treatment of other disorders that can be treated using fatty acid amides or modulators of cannabinoid receptors, such as, for example, anxiety, eating disorders, metabolic disorders, cardiovascular disorders, and inflammation.

The various fatty acid ethanolamides have important and diverse physiological functions. As a result, inhibitor molecules that selectively inhibit FAAH enzymatic activity would allow a corresponding selective modulation of the cellular and extra-cellular concentrations of a FAAH substrate. FAAH inhibitors that are biologically compatible could be effective pharmaceutical compounds when formulated as therapeutic agents for any clinical indication where FAAH enzymatic inhibition is desired. In some embodiments, FAAH activity in peripheral tissues can be preferentially inhibited. In some embodiments, FAAH inhibitors that do substantially cross the blood-brain-barrier can be used to preferentially inhibit FAAH activity in peripheral tissues. In some embodiments, FAAH inhibitors that preferentially inhibit FAAH activity in peripheral tissues can minimize the effects of FAAH inhibition in the central nervous system. In some embodiments, it is preferred to inhibit FAAH activity in peripheral tissues and minimize FAAH inhibition in the central nervous system.

Diseases, disorders, syndromes and/or conditions, that would benefit from inhibition of FAAH enzymatic activity include, for example, Alzheimer's Disease, schizophrenia, depression, alcoholism, addiction, suicide, Parkinson's disease, Huntington's disease, stroke, emesis, miscarriage, embryo implantation, endotoxic shock, liver cirrhosis, atherosclerosis, cancer, traumatic head injury, glaucoma, and bone cement implantation syndrome.

Other diseases, disorders, syndromes and/or conditions that would benefit from inhibition of FAAH activity, include, for example, multiple sclerosis, retinitis, amyotrophic lateral sclerosis, immunodeficiency virus-induced encephalitis, attention-deficit hyperactivity disorder, pain, nociceptive pain, neuropathic pain, inflammatory pain, non-inflammatory pain, painful hemorrhagic cystitis, obesity, hyperlipidemia, metabolic disorders, feeding and fasting, alteration of appetite, stress, memory, aging, hypertension, septic shock, cardiogenic shock, intestinal inflammation and motility, irritable bowel syndrome, colitis, diarrhea, ileitis, ischemia, cerebral ischemia, hepatic ischemia, myocardial infarction, cerebral excitotoxicity, seizures, febrile seizures, neurotoxicity, neuropathies, sleep, induction of sleep, prolongation of sleep, insomnia, and inflammatory diseases.

Neurological and psychological disorders that would benefit from inhibition of FAAH activity include, for example, pain, depression, anxiety, generalized anxiety disorder (GAD), obsessive compulsive disorders, stress, stress urinary incontinence, attention deficit hyperactivity disorders, schizophrenia, psychosis, Parkinson's disease, muscle spasticity, epilepsy, diskenesia, seizure disorders, jet lag, and insomnia.

FAAH inhibitors can also be used in the treatment of a variety of metabolic syndromes, diseases, disorders and/or conditions, including but not limited to, insulin resistance syndrome, diabetes, hyperlipidemia, fatty liver disease, obesity, atherosclerosis and arteriosclerosis.

FAAH inhibitors are useful in the treatment of a variety of painful syndromes, diseases, disorders and/or conditions, including but not limited to those characterized by non-inflammatory pain, inflammatory pain, peripheral neuropathic pain, central pain, deafferentiation pain, chronic nociceptive pain, stimulus of nociceptive receptors, phantom and transient acute pain.

Inhibition of FAAH activity can also be used in the treatment of a variety of conditions involving inflammation. These conditions include, but are not limited to arthritis (such as rheumatoid arthritis, shoulder tendonitis or bursitis, gouty arthritis, and aolymyalgia rheumatica), organ-specific inflammatory diseases (such as thyroiditis, hepatitis, inflammatory bowel diseases), asthma, other autoimmune diseases (such as multiple sclerosis), chronic obstructive pulmonary disease (COPD), allergic rhinitis, and cardiovascular diseases.

In some cases, FAAH inhibitors are useful in preventing neurodegeneration or for neuroprotection.

In addition, it has been shown that when FAAH activity is reduced or absent, one of its substrates, anandamide, acts as a substrate for COX-2, which converts anandamide to prostamides (Weber et al. J. Lipid. Res. 2004; 45:757). Concentrations of certain prostamides may be elevated in the presence of a FAAH inhibitor. Certain prostamides are associated with reduced intraocular pressure and ocular hypotensivity. Thus, in one embodiment, FAAH inhibitors may be useful for treating glaucoma.

In some embodiments, FAAH inhibitors can be used to treat or reduce the risk of EMDs, which include, but are not limited to, obesity, appetite disorders, overweight, cellulite, Type I and Type II diabetes, hyperglycemia, dyslipidemia, steatohepatitis, liver steatosis, non-alcoholic steatohepatitis, Syndrome X, insulin resistance, diabetic dyslipidemia, anorexia, bulimia, anorexia nervosa, hyperlipidemia, hypertriglyceridemia, atherosclerosis, arteriosclerosis, inflammatory disorders or conditions, Alzheimers disease, Crohn's disease, vascular inflammation, inflammatory bowel disorders, rheumatoid arthritis, asthma, thrombosis, or cachexia.

In other embodiments, FAAH inhibitors can be used to treat or reduce the risk of insulin resistance syndrome and diabetes, i.e., both primary essential diabetes such as Type I Diabetes or Type II Diabetes and secondary nonessential diabetes. Administering a composition containing a therapeutically effective amount of an in vivo FAAH inhibitor reduces the severity of a symptom of diabetes or the risk of developing a symptom of diabetes, such as atherosclerosis, hypertension, hyperlipidemia, liver steatosis, nephropathy, neuropathy, retinopathy, foot ulceration, or cataracts.

In another embodiment, FAAH inhibitors can be used to treat food abuse behaviors, especially those liable to cause excess weight, e.g., bulimia, appetite for sugars or fats, and non-insulin-dependent diabetes.

In some embodiments, FAAH inhibitors can be used to treat a subject suffering from an EMD and also suffers from a depressive disorder or from an anxiety disorder. Preferably, the subject is diagnosed as suffering from the depressive or psychiatric disorder prior to administration of the FAAH inhibitor composition. Thus, a dose of a FAAH inhibitor that is therapeutically effective for both the EMD and the depressive or anxiety disorder is administered to the subject. Methods for treatment of anxiety and depressive disorders by FAAH inhibition are described in, e.g., U.S. patent application Ser. Nos. 10/681,858 and 60/755,035.

Preferably, the subject to be treated is human. However, the methods can also be used to treat non-human mammals. Animal models of EMDs such as those described in, e.g., U.S. Pat. No. 6,946,491 are particularly useful.

Symptoms, diagnostic tests, and prognostic tests for each of the above-mentioned conditions are known in the art. See, e.g., “Harrison's Principles of Internal Medicine©,” 16th ed., 2004, The McGraw-Hill Companies, Inc., and the “Diagnostic and Statistical Manual of Mental Disorders©,” 4th ed., 1994, American Psychiatric Association.

FAAH inhibitor compositions can also be used to decrease body-weight in individuals wishing to decrease their body weight for cosmetic, but not necessarily medical considerations.

A FAAH inhibitor composition can be administered in combination with a drug for lowering circulating cholesterol levels (e.g., statins, niacin, fibric acid derivatives, or bile acid binding resins). FAAH inhibitor compositions can also be used in combination with a weight loss drug, e.g., orlistat or an appetite suppressant such as diethylpropion, mazindole, orlistat, phendimetrazine, phentermine, or sibutramine.

The methods described herein can also include providing an exercise regimen or providing a calorie-restricted diet (e.g., a triglyceride-restricted diet) to the subject.

Esters of alkylcarbamic acids and alkylthiocarbamic acids have shown promise as selective FAAH inhibitors (Kathuria et al., Nat. Med. 2003, 9:76-81). A series of alkylcarbamic acid aryl esters, such as, for example, cyclohexylcarbamic acid 3′-carbamoylbiphenyl-3-yl ester (also known as 5′-carbamoylbiphenyl-3-yl cyclohexyl carbamate, UCM597, URB597, and KDS-4103 (URB-597)), have been shown to be potent and selective inhibitors of FAAH activity. Alkylcarbamic acid aryl esters, such as, for example, cyclohexylcarbamic acid 3′-carbamoylbiphenyl-3-yl ester, have been shown to be potent and selective inhibitors of FAAH activity, which do not significantly interact with selected serine hydrolases or with cannabinoid receptors (Mor et al. J. Med. Chem. 2004, 47:4998-5008; Piomelli et al. International Patent Publication No. WO 2004/033422; incorporated by reference).

Alkylcarbamic acid aryl esters inhibit FAAH activity through an irreversible interaction with FAAH, possibly due to a nucleophilic attack of an active serine residue (Serine 241) of FAAH on the carbamate moiety of the alkylcarbamic acid aryl ester compounds (Kathuria et al Nature Medicine, vol. 9, no. 1, 76-81, 2003; Deutsch et al. Prostaglandins, Luekotrienes and Essential Fatty Acids (2002) 66(2&3), 201-210; Alexander et al. Chemistry & Biology, vol. 12, 1179-1187; 2005.). Metabolism of the alkylcarbamic acid aryl ester inhibitors by the FAAH enzyme results in the hydrolysis of the carbamate compounds and release of the aryloxy portion of the alkylcarbamic acid aryl ester inhibitor.

Provided herein are compound, which are esters of alkylcarbamic acids, compositions that include them, and methods of their use. Compounds provided herein have a structure selected from among:

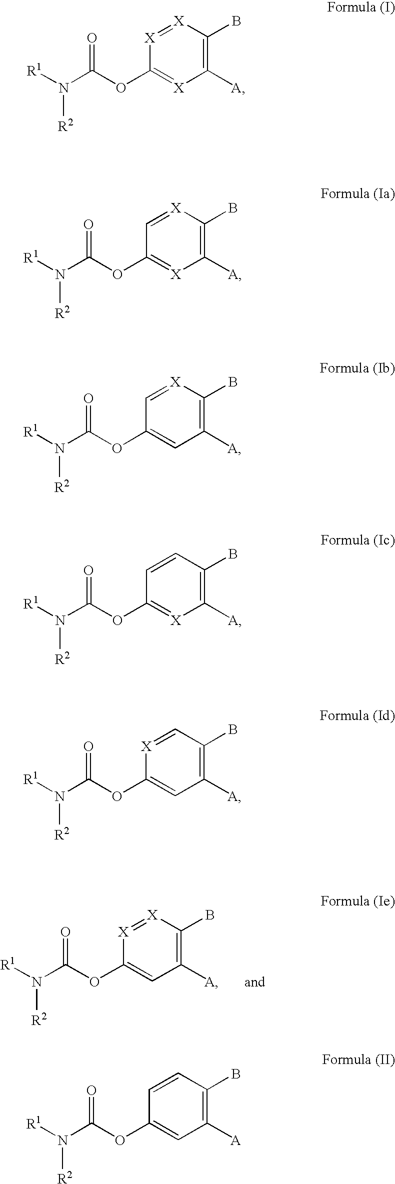

wherein:
R¹ is an optionally substituted group selected from among C₁-C₆ alkyl, C₃-C₉ cycloalkyl and —C₁-C₄alkyl(C₃-C₉cycloalkyl);
R² is H or an optionally substituted alkyl;
if R¹ is an optionally substituted C₃-C₉cycloalkyl or an optionally substituted —C₁-C₄alkyl(C₃-C₉cycloalkyl), then any carbon of the R¹ carbocyclic ring can be optionally substituted by both Y and Z;
Y and Z are independently selected from among halogen or methyl; or
Y and Z taken together form an oxo (═O);
one of A or B is -L-G and the other is selected from among H and an optionally substituted C₁-C₆ alkyl; or
A and B are both selected from among H and an optionally substituted C₁-C₆ alkyl provided that at least one X is present and is N;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is tetrazolyl, —NHS(═O)₂R⁸, —S(═O)₂NHR⁹, —OH, —SH, —C(O)NHC(O)R⁸, —C(O)NHS(═O)₂R⁸, —S(═O)₂NHC(O)R⁸, —S(═O)₂NHC(O)NHR⁸, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R)₂, —CO₂H, a carboxylic acid bioisostere, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)ₙOH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
X is CH or N, provided that at least one X is N;
n is 1, 2, 3, or 4; x is 1, 2, or 3; y is 0, 1, or 2; and
   pharmaceutically acceptable salts, pharmaceutically acceptable N-oxides, pharmaceutically active metabolites, pharmaceutically acceptable prodrugs, or pharmaceutically acceptable solvates thereof.

### Certain Chemical Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

An “alkoxy” group refers to a (alkyl)O— group, where alkyl is as defined herein.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group).

As used herein, C₁-Cₓ includes C₁-C₂, C₁-C₃ . . . C₁-Cₓ.

The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

As used herein, the term “non-cyclic alkyl” refers to an alkyl that is not cyclic (i.e., a straight or branched chain containing at least one carbon atom). Non-cyclic alkyls can be fully saturated or can contain non-cyclic alkenes and/or alkynes. Non-cyclic alkyls can be optionally substituted.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which may be the same or different. Non-limiting examples of an alkenyl group include —CH═CH₂, —C(CH₃)═CH₂, —CH═CHCH₃ and —C(CH₃)═CHCH₃. The alkenyl moiety may be branched, straight chain, or cyclic (in which case, it would also be known as a “cycloalkenyl” group). Depending on the structure, an alkenyl group can be a monoradical or a diradical (i.e., an alkenylene group). Alkenyl groups can be optionally substituted.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CH₃ and —C≡CH₂CH₃. The “R” portion of the alkynyl moiety may be branched, straight chain, or cyclic. Depending on the structure, an alkynyl group can be a monoradical or a diradical (i.e., an alkynylene group). Alkynyl groups can be optionally substituted.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “aromatic” refers to a planar ring having a delocalized π-electron system containing 4n+2 π electrons, where n is an integer. Aromatic rings can be formed by five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroarornatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

The term “bond” or “single bond” refers to a chemical bond between two atoms, or two moieties when the atoms joined by the bond are considered to be part of larger substructure.

The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

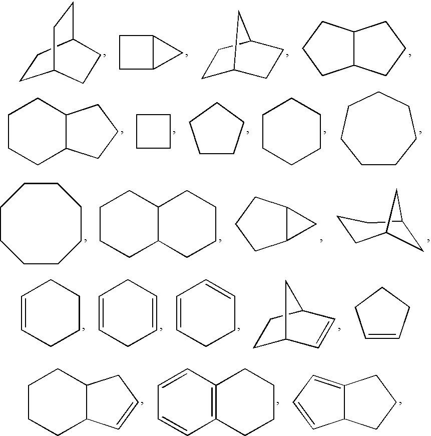

and the like. Depending on the structure, an cycloalkyl group can be a monoradical or a diradical (e.g., an cycloalkylene group).

As used herein, the term “carbocycle” refers to a ring, wherein each of the atoms forming the ring is a carbon atom. Carbocylic rings can be formed by three, four, five, six, seven, eight, nine, or more than nine carbon atoms. Carbocycles can be optionally substituted.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “halo” or, alternatively, “halogen” or “halide” means fluoro, chloro, bromo or iodo.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

As used herein, the terms “heteroalkyl” “heteroalkenyl” and “heteroalkynyl” include optionally substituted alkyl, alkenyl and alkynyl radicals in which one or more skeletal chain atoms are selected from an atom other than carbon, e.g., oxygen, nitrogen, sulfur, silicon, phosphorus or combinations thereof.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can form part of a ring system.

As used herein, the term “ring system” refers to two or more rings, wherein two or more of the rings are fused. The term “fused” refers to structures in which two or more rings share one or more bonds.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

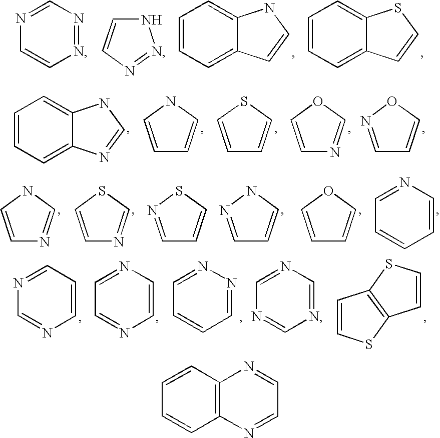

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

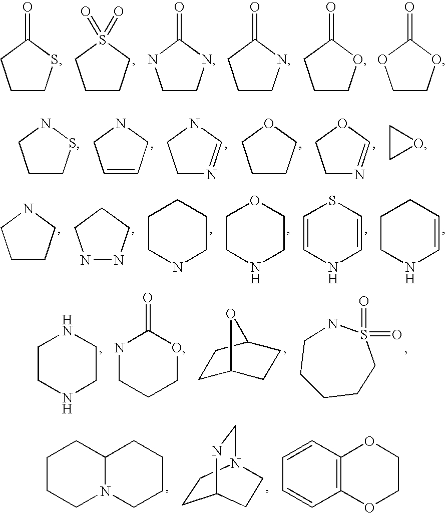

and the like. The term heteroalicyclic also includes all ring forms of the carbohydrates, including but not limited to the monosaccharides, the disaccharides and the oligosaccharides.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

As used herein, “carboxylic acid bioisostere” refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisoteres of a carboxylic acid include, but are not limited to,

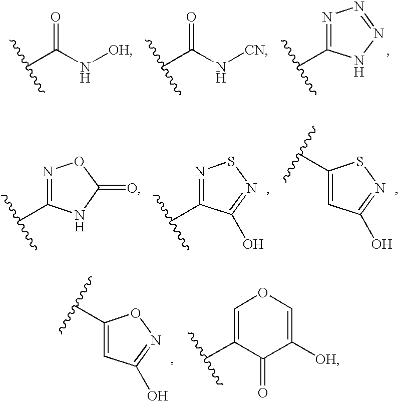

and the like.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

An “isocyanato” group refers to a —NCO group.

An “isothiocyanato” group refers to a —NCS group.

The term “moiety” refers to a specific segment or functional group of a molecule. Chemical moieties are often recognized chemical entities embedded in or appended to a molecule.

A “sulfinyl” group refers to a —S(═O)—R.

A “sulfonyl” group refers to a —S(═O)₂—R.

A “thioalkoxy” group refers to a —S-alkyl group.

As used herein, the term “O-carboxy” refers to a group of formula RC(═O)O—.

As used herein, the term “C-carboxy” refers to a group of formula —C(═O)OR.

As used herein, the term “acetyl” refers to a group of formula —C(═O)CH₃.

As used herein, the term “trihalomethanesulfonyl” refers to a group of formula X₃CS(═O)₂— where X is a halogen.

As used herein, the term “cyano” refers to a group of formula —CN.

As used herein, the term “S-sulfonamido” refers to a group of formula —S(═O)₂NR₂.

As used herein, the term “N-sulfonamido” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “O thiocarbamyl” refers to a group of formula —OC(═S)NR₂.

As used herein, the term “N thiocarbamyl” refers to a group of formula ROC(═S)NH—.

As used herein, the term “C-amido” refers to a group of formula —C(═O)NR₂.

As used herein, the term “N-amido” refers to a group of formula RC(═O)NH—.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, — (substituted or unsubstituted C₁-C₆ alkyl), or -(substituted or unsubstituted C₂-C₆ alkenyl); and each Rₛ is independently selected from H, (substituted or unsubstituted lower alkyl), (substituted or unsubstituted lower cycloalkyl), heteroaryl, or heteroalkyl. The protecting groups that may form the protective derivatives of the above substituents are known to those of skill in the art and may be found in references such as Greene and Wuts, above.

The compounds presented herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof can be chosen by one skilled in the field to provide stable moieties and compounds.

### Compounds

Certain compounds that inhibit the activity of fatty acid amide hydrolase (FAAH) play a role in health. In certain embodiments, FAAH inhibitor compounds are useful in treating any of a variety of diseases, disorders or conditions. In certain embodiments, compounds provided herein are selective FAAH inhibitor compounds.

FAAH inhibitor compositions have been described in U.S. patent application Ser. Nos. 10/681,858, 60/755,035; U.S. Pat. Nos. 6,462,054, 6,949,574 and 6,891,043; International Patent Publication No. WO 04020430, WO 04067498, WO 04099176, WO 05033066, WO 02087569, WO 03065989, WO 9749667, WO 9926584, WO 04033652, and WO 06044617; Cravatt et al. Current Opinion in Chemical Biology, 2003, 7:469-475.; Kathuria et al. Nature Medicine, vol. 9, no. 1, pp 76-81, 2003.; Tarzia et al. J. Med. Chem. 2003, 46, 2352-2360.; and Drysdale et al. Current Medicinal Chemistry, 2003, 10, 2719-2732.

Described herein are compounds that inhibit the activity of FAAH. Also described herein are pharmaceutically acceptable salts, pharmaceutically active metabolites and pharmaceutically acceptable prodrugs of such compounds. Pharmaceutical compositions that include at least one such compound or a pharmaceutically acceptable salt, pharmaceutically active metabolite or pharmaceutically acceptable prodrug of such compound, are provided.

In some embodiments, compounds provided herein are ionizable and do not substantially cross the blood brain barrier. In some embodiments, provided herein are carbamate FAAH inhibitors that are ionizable at physiological pH, and therefore less likely to cross the blood brain barrier. In some embodiments, compounds provided herein have a moiety that is ionizable at physiological pH. In other embodiments, compounds provided herein have a charge at physiological pH. In some other embodiments, compounds provided herein are protonated at physiological pH. In other embodiments, compounds provided herein are deprotonated at physiological pH. Such FAAH inhibitors are particularly useful when it is desirable to minimize and/or avoid psychotropic effects caused by FAAH inhibition in the central nervous system.

In some embodiments, compounds provided herein have a structure selected from among:

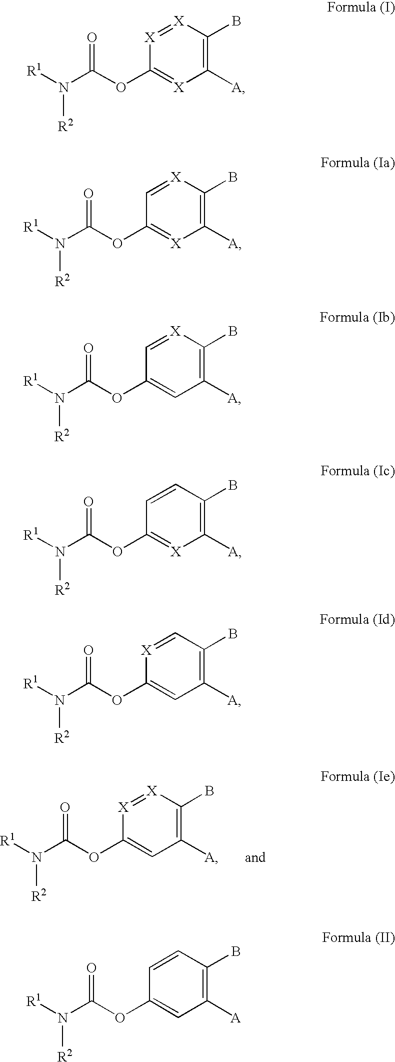

wherein:
R¹ is an optionally substituted group selected from among C₁-C₆ alkyl, C₃-C₉ cycloalkyl and —C₁-C₄alkyl(C₃-C₉cycloalkyl);
R² is H or an optionally substituted alkyl;
one of A or B is -L-G and the other is selected from among H and an optionally substituted C₁-C₆ alkyl; or
A and B are both H provided that at least one X present and is N;
L is a bond, or an optionally substituted group selected from among C₁-C₆ alkylene, C₁-C₆ heteroalkylene, C₁-C₆ ketoalkylene, —C(O)NR₉—(CH₂)ₙ—, —NR₉—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —NR⁹S(═O)₂R⁸, —S(═O)₂N(R)₂, —OR⁹, —OC(O)N(R⁹)₂, —NR⁹C(O)OR⁸, —C(O)NHS(═O)₂R⁸, —S(═O)₂NHC(O)R⁸, —N(R⁹)₂, —N(R⁹)C(O)R⁸, —NR⁹C(O)N(R⁹)₂, —C(═NR¹⁰)N(R⁹)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —C(R⁹)₂(OR⁹), —CO₂R⁹, —CON(R⁹)₂, —OS(═O)₂OR⁹, —OP(═O)₂OR⁹, or -L⁵-(substituted or unsubstituted heteroaryl containing 1-3 N atoms);
L⁵ is —OC(O)O—, —NHC(O)NH—, —NHC(O)O—, —O(O)CNH—, —NHC(O)—, —C(O)NH—, —C(O)O—, or —OC(O)—;
each R⁸ is independently a substituted or unsubstituted C₁-C₆ alkyl;
each R⁹ is independently H, a substituted C₁-C₆ alkyl or unsubstituted C₁-C₆ alkyl; and
each R¹⁰ is independently selected from among H, —S(═O)₂R⁸, —S(═O)₂NH₂—C(O)R⁸, —CN, and —NO₂;
X is CH or N;
n is 1, 2, 3, or 4; and
   pharmaceutically acceptable salts, pharmaceutically acceptable N-oxides, pharmaceutically active metabolites, pharmaceutically acceptable prodrugs, or pharmaceutically acceptable solvates thereof.

In some embodiments, compounds provided herein have a structure according to Formula (I)

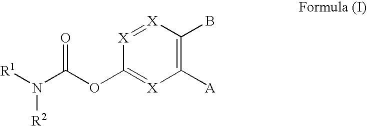

wherein:
R¹ is an optionally substituted group selected from among C₁-C₆ alkyl, C₃-C₉ cycloalkyl and —C₁-C₄alkyl(C₃-C₉cycloalkyl);
R² is H or an optionally substituted alkyl; one of A or B is -L-G and the other is selected from among H and an optionally substituted C₁-C₆ alkyl, A and B can together form a ring structure; or
A and B are both H provided that at least one X is N;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —NR⁹S(═O)₂R⁸, —S(═O)₂N(R⁹)₂, —OR⁹, —OC(O)N(R⁹)₂, —NR⁹C(O)OR⁸, —C(O)NHS(═O)₂R⁸, —S(═O)₂NHC(O)R⁸, —N(R⁹)₂, —N(R⁹)C(O)R⁸, —NR⁹C(O)N(R⁹)₂, —C(═NR¹⁰)N(R⁹)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —C(R⁹)₂(OR⁹), —CO₂R⁹, —CON(R⁹)₂, —OS(═O)₂OR⁹, —OP(═O)₂OR⁹, or -L⁵-(substituted or unsubstituted heteroaryl containing 1-3 N atoms);
L⁵ is —OC(O)O—, —NHC(O)NH—, —NHC(O)O—, —O(O)CNH—, —NHC(O)—, —C(O)NH—, —C(O)O—, or —OC(O)—;
each R⁸ is independently a substituted or unsubstituted C₁-C₆ alkyl;
each R⁹ is independently H, a substituted C₁-C₆ alkyl or unsubstituted C₁-C₆ alkyl; and
each R¹⁰ is independently selected from among H, —S(═O)₂R⁸, —S(═O)₂NH₂—C(O)R⁸, —CN, and —NO₂;
X is CH or N;
n is 1, 2, 3, or 4; and
   pharmaceutically acceptable salts, pharmaceutically acceptable N-oxides, pharmaceutically active metabolites, pharmaceutically acceptable prodrugs, or pharmaceutically acceptable solvates thereof.

In some embodiments, compounds provided herein have a structure of Formula (Ia):

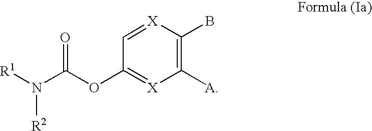

In other embodiments, compounds provided herein have a structure of Formula (Ib):

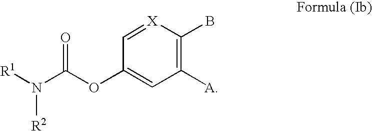

In some other embodiments, compounds provided herein have a structure of Formula (Ic):

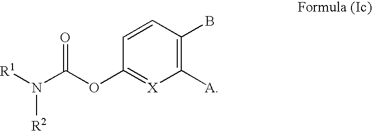

In another embodiment, compounds provided herein have a structure of Formula (Id):

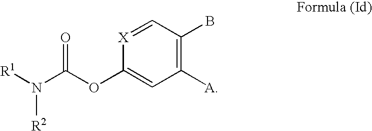

In yet some other embodiments, compounds provided herein have a structure of Formula (Ie):

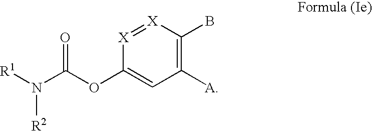

In some embodiments, both A and B are H provided that at least one X is present and is N. In embodiments where X is present, at least one X is N.

In some embodiments, provided herein are compounds that have a structure of Formula (II):

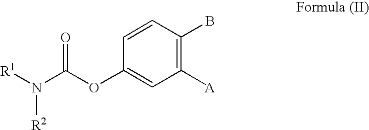

wherein:
R¹ is an optionally substituted group selected from among C₁-C₆ alkyl, C₃-C₉ cycloalkyl and —C₁-C₄alkyl(C₃-C₉cycloalkyl);
R² is H or an optionally substituted alkyl;

one of A or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, —C(O)NR₉—(CH₂)ₙ—, —NR₉—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —NR⁹S(═O)₂R⁸, —S(═O)₂N(R⁹)₂, —OR⁹, —OC(O)N(R⁹)₂, —NR⁹C(O)OR⁸, —C(O)NHS(═O)₂R⁸, —S(═O)₂NHC(O)R¹⁰, —N(R⁹)₂, —N(R⁹)C(O)R⁸, —NR⁹C(O)N(R⁹)₂, —C(—NR¹⁰)N(R⁹)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —C(R⁹)₂(OR⁹), —CO₂R⁹, —CON(R⁹)₂, —OS(═O)₂OR⁹, —OP(═O)₂OR⁹, or -L⁵-(substituted or unsubstituted heteroaryl containing 1-3 N atoms);
L⁵ is —OC(O)O—, —NHC(O)NH—, —NHC(O)O—, —O(O)CNH—, —NHC(O)—, —C(O)NH—, —C(O)O—, or —OC(O)—;
each R⁸ is independently a substituted or unsubstituted C₁-C₆ alkyl;
each R⁹ is independently H, a substituted C₁-C₆ alkyl or unsubstituted C₁-C₆ alkyl; and
each R¹⁰ is independently selected from among H, —S(═O)₂R⁸, —S(═O)₂NH₂—C(O)R⁸, —CN, and —NO₂;
n is 1, 2, 3, or 4; and
   pharmaceutically acceptable salts, pharmaceutically acceptable N-oxides, pharmaceutically active metabolites,
   pharmaceutically acceptable prodrugs, or pharmaceutically acceptable solvates thereof.

For any and all of the embodiments, substituents can be selected from among a subset of the listed alternatives. For example, in some embodiments, one of A or B is -L-G and the other is H. In some embodiments, A is -L-G. In other embodiments, B is -L-G.

In some embodiments, R² is H.

In some embodiments, L is a bond, or an optionally substituted group selected from among C₁-C₆ alkylene, C₁-C₆ ketoalkylene, —C(O)NR₉—(CH₂)ₙ—, —NR₉—C(O)—(CH₂)ₙ—, —NR⁹C(O)N(R)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—. In other embodiments, L is a bond, or an optionally substituted group selected from among C₁-C₆ alkylene, C₁-C₆ ketoalkylene, —C(O)NR₉—(CH₂)ₙ—, —NR₉—C(O)—(CH₂)ₙ—, —NR⁹C(O)N(R⁹)—(CH₂)ₙ—, —S(O)—(CH₂)ₙ—, and —S(O)₂—(CH₂)ₙ—. In some other embodiments, L is a bond, or an optionally substituted group selected from among C₁-C₆ alkyl and C₁-C₆ ketoalkyl. In yet other embodiments, L is a bond.

In some embodiments, G is H, tetrazolyl, —NR⁹S(═O)₂R⁸, —S(═O)₂N(R⁹)₂, —OR⁹, —OC(O)N(R⁹)₂, —NR⁹C(O)OR⁸, —C(O)NHS(═O)₂R⁸, —S(═O)₂NHC(O)R⁸, —N(R⁹)₂, —N(R⁹)C(O)R⁸, —NR⁹C(O)N(R⁹)₂, —C(═NR¹⁰)N(R⁹)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂R⁹, —CON(R⁹)₂, —OS(═O)₂OR⁹, or —OP(═O)₂OR⁹. In other embodiments, G is H, tetrazolyl, —NR⁹S(═O)₂R⁸, —S(═O)₂N(R⁹)₂, —OR⁹, —C(O)NHS(═O)₂R⁸, —S(═O)₂NHC(O)R⁸, —N(9)₂, —N(R⁹)C(O)R⁸, —C(═NR¹⁰)N(R⁹)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂R⁹, or —CON(R⁹)₂. In some other embodiments, G is H, tetrazolyl, —NR⁹S(═O)₂R⁸, —S(═O)₂N(R⁹)₂, —OR⁹, —C(O)NHS(═O)₂R⁸, —S(═O)₂NHC(O)R⁸, —N(R⁹)₂, —N(R⁹)C(O)R⁸, —C(═NR¹⁰)N(R⁹)₂, —NR⁹C(═NR¹⁰)N(R⁹)₂, —CO₂R⁹, or —CON(R⁹)₂. In yet some other embodiments, G is H, tetrazolyl, —NR⁹S(═O)₂R⁸, —S(═O)₂N(R⁹)₂, —OR⁹, —C(O)NHS(═O)₂R⁸, —S(═O)₂NHC(O)R⁸, —N(R⁹)₂, —N(R⁹)C(O)R⁸, —C(═NR¹⁰)N(R⁹)₂, —NR⁹C(═NR¹⁰)N(R⁹)₂, —CO₂R⁹, or —CON(R⁹)₂; each R⁸ is independently a substituted or unsubstituted C₁-C₆ alkyl; each R⁹ is H; and each R¹⁰ is independently selected from among H, —S(═O)₂R⁸, —S(═O)₂NH₂, and —C(O)R⁸.

In some embodiments, G is -L⁵-(substituted or unsubstituted heteroaryl containing 1-3 N atoms); and L⁵ is —OC(O)O—, —NHC(O)NH—, —NHC(O)O—, —O(O)CNH—, —NHC(O)—, —C(O)NH—, —C(O)O—, or —OC(O)—. In other embodiments, G is -L⁵-(substituted or unsubstituted heteroaryl containing 1-3 N atoms); and L⁵ is a bond, —OC(O)O—, —NHC(O)NH—, —NHC(O)O—, —O(O)CNH—, —NHC(O)—, —C(O)NH—, —C(O)O—, or —OC(O)—. In some other embodiments, G is -L⁵-(substituted or unsubstituted heteroaryl containing 1-3 N atoms); and L⁵ is a bond. In yet other embodiments, L is a bond, G is -L⁵-(substituted or unsubstituted heteroaryl containing 1-3 N atoms); and L⁵ is a bond.

In some embodiments, G is not H.

In some embodiments, compounds provided herein have a structure of Formula (II):

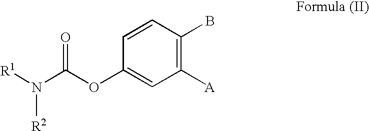

wherein:
R¹ is an optionally substituted group selected from among C₁-C₆ alkyl, C₃-C₉ cycloalkyl and —C₁-C₄alkyl(C₃-C₉cycloalkyl);
R² is H or an optionally substituted alkyl; one of A or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —NHS(═O)₂R⁸, —S(═O)₂NHR⁹, —S(═O)₂NH-phenyl, —OH, —SH, —OC(O)NHR⁹, —NHC(O)OR⁸, —C(O)NHC(O)R⁸, —C(O)NHS(═O)₂R⁸, —S(═O)₂NHC(O)R⁸, —S(═O)₂NHC(O)NHR⁹, —N(R⁹)₂, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —OS(═O)₂OH, —OP(═O)₂OH, or -L⁵-(substituted or unsubstituted heteroaryl containing 1-3 N atoms);
L⁵ is a bond, —OC(O)O—, —NHC(O)NH—, —NHC(O)O—, —O(O)CNH—, —NHC(O)—, —C(O)NH—, —C(O)O—, or —OC(O)—;
each R⁸ is independently a substituted or unsubstituted C₁-C₆ alkyl;
each R⁹ is independently H, a substituted C₁-C₆ alkyl or unsubstituted C₁-C₆ alkyl; and
each R¹⁰ is independently selected from among H, —S(═O)₂R⁸, —S(═O)₂NH₂, —C(O)R⁸, —CN, and —NO₂;
n is 1, 2, 3, or 4; and
pharmaceutically acceptable salts, pharmaceutically acceptable N-oxides, pharmaceutically active metabolites, pharmaceutically acceptable prodrugs, or pharmaceutically acceptable solvates thereof.

For any and all of the embodiments, substituents can be selected from among from a subset of the listed alternatives. For example, in some embodiments, one of A or B is -L-G and the other is H or an optionally substituted C₁-C₆ alkyl. In other embodiments, one of A or B is -L-G and the other is H. In some embodiments, A is -L-G. In some other embodiments, B is -L-G.

In certain embodiments, R² is H.

In some embodiments, L is a bond, or an optionally substituted group selected from among C₁-C₆ alkylene, C₁-C₆ ketoalkylene, —C(O)NR⁹—(CH₂)ₙ—, —NR⁹—C(O)—(CH₂)ₙ—, —NR⁹C(O)N(R⁹)—(CH₂)ₙ—, —S(O)—(CH₂)ₙ—, —S(O)₂—(CH₂)ₙ—, C(═NR¹⁰)N(R⁹)—(CH₂)ₙ—, and —NR⁹C(═NR¹⁰)N(R⁹)—(CH₂)ₙ—. In other embodiments, L is a bond, or an optionally substituted group selected from among C₁-C₆ alkylene, C₁-C₆ ketoalkylene, —C(O)NR₉—(CH₂)ₙ—, —NR₉—C(O)—(CH₂)ₙ—, —NR⁹C(O)N(R)—(CH₂)ₙ—, —S(O)—(CH₂)ₙ—, and —S(O)₂—(CH₂)ₙ—. In some other embodiments, L is a bond, or an optionally substituted group selected from among C₁-C₆ alkylene, C₁-C₆ ketoalkylene, —C(O)NR₉—(CH₂)ₙ—, —NR₉—C(O)—(CH₂)ₙ—, and —NR⁹C(O)N(R⁹)—(CH₂)ₙ—. In other embodiments, L is a bond, or an optionally substituted group selected from among C₁-C₆ alkyl and C₁-C₆ ketoalkyl. In some embodiments, L is a bond.

In some embodiments, G is H, tetrazolyl, —NHS(═O)₂R⁸, —S(═O)₂NHR⁹, —S(═O)₂NH-phenyl, —OH, —SH, —OC(O)NHR⁹, —NHC(O)OR⁸, —C(O)NHC(O)R⁸, —C(O)NHS(═O)₂R⁸, —S(═O)₂NHC(O)R⁸, —S(═O)₂NHC(O)NHR⁹, —N(R⁹)₂, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —OS(═O)₂OH, or —OP(═O)₂OH.

In other embodiments, G is H, tetrazolyl, —NHS(═O)₂R⁸, —S(═O)₂NHR⁹, —S(═O)₂NH-phenyl, —OH, —C(O)NHC(O)R⁸, —C(O)NHS(═O)₂R⁸, —S(═O)₂NHC(O)R⁸, —S(═O)₂NHC(O)NHR⁹, —N(R⁹)₂, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R)₂, or —CO₂H. In some other embodiments, G is H, tetrazolyl, —NHS(═O)₂R⁸, —S(═O)₂NHR⁹, —S(═O)₂NH-phenyl, —OH, —C(O)NHC(O)R⁸, —C(O)NHS(═O)₂R⁸, —S(═O)₂NHC(O)R⁸, —S(═O)₂NHC(O)NHR⁹, —N(R⁹)₂, —NHC(O)R⁸, —NHC(O)N(R⁹)₂, —C(═NR¹⁰)N(R⁹)₂, —NR⁹C(═NR¹⁰)N(R⁹)₂, or —CO₂H. In yet other embodiments, G is H, tetrazolyl, —NHS(═O)₂R⁸, —S(═O)₂NHR⁹, —S(═O)₂NH-phenyl, —OH, —C(O)NHS(═O)₂R⁸, —S(═O)₂NHC(O)R⁸, —N(R⁹)₂, —NHC(O)R⁸, —NHC(O)N(R⁹)₂, —C(═NR¹⁰)N(R⁹)₂, —NR⁹C(═NR¹⁰)N(R⁹)₂, or —CO₂H; each R⁸ is independently a substituted or unsubstituted C₁-C₆ alkyl; each R⁹ is H; and each R¹⁰ is independently selected from among H, —S(═O)₂R⁸, —S(═O)₂NH₂, and —C(O)R⁸.

In some embodiments, each R⁸ is independently a substituted or unsubstituted C₁-C₆ alkyl; each R⁹ is H; and

each R¹⁰ is independently selected from among H, —S(═O)₂R⁸, and —C(O)R⁸. In some embodiments, each R⁹ is H.

In some embodiments, G is -L 5-(substituted or unsubstituted heteroaryl containing 1-3 N atoms); and L⁵ is a bond, —OC(O)O—, —NHC(O)NH—, —NHC(O)O—, —O(O)CNH—, —NHC(O)—, —C(O)NH—, —C(O)O—, or —OC(O)—. In some embodiments, G is -L⁵-(substituted or unsubstituted heteroaryl containing 1-3 N atoms); and L⁵ is a bond. In some embodiments, G is not H.

In some embodiments, R¹ is an optionally substituted group selected from among C₃-C₉cycloalkyl, and —C₁-C₄alkyl-(C₃-C₉cycloalkyl). In other embodiments, R¹ is an optionally substituted C₃-C₉cycloalkyl. In some other embodiments, R¹ is an optionally substituted —C₁-C₄alkyl-(C₃-C₉cycloalkyl).

In some embodiments, R¹ is selected from among methyl, ethyl, propyl, isopropyl, n-butyl, sec-butyl, iso-butyl, tert-butyl, pentyl, hexyl, cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl. In other embodiments, R¹ is selected from among isopropyl, sec-butyl, iso-butyl, tert-butyl, cyclopropyl, cyclobutyl, cyclopentyl, cyclohexyl, cycloheptyl, cyclopropylmethyl, cyclobutylmethyl, cyclopentylmethyl, cyclohexylmethyl and cycloheptylmethyl. In some other embodiments, R¹ is selected from among isopropyl, sec-butyl, iso-butyl, tert-butyl, cyclopropyl, cyclopentyl, cyclohexyl, cyclopropylmethyl, cyclopentylmethyl, and cyclohexylmethyl. In yet other embodiments, R¹ is selected from among isopropyl, sec-butyl, iso-butyl, cyclohexyl, cyclopropylmethyl and cyclohexylmethyl. In some embodiments, R¹ is selected from among cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl. In other embodiments provided herein, R¹ is selected from among cyclohexyl and cyclohexylmethyl.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments, compounds provided herein have a structure selected from among:

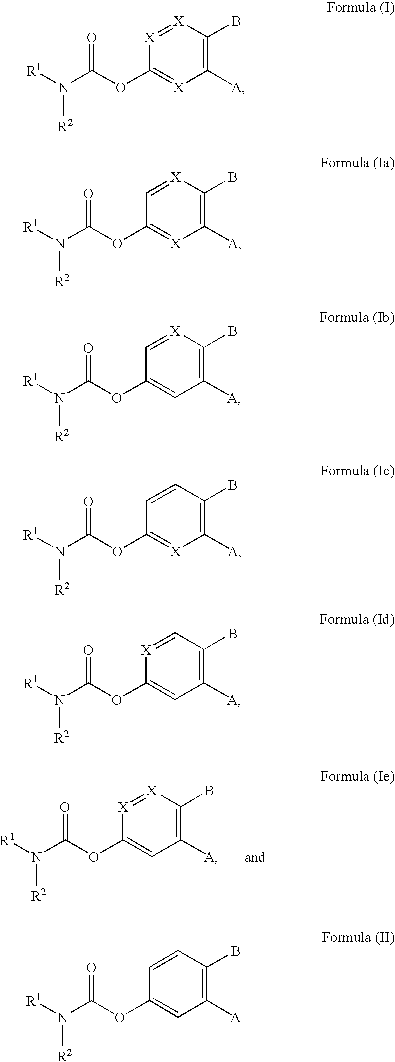

wherein:
R¹ is an optionally substituted group selected from among C₁-C₆ alkyl, C₃-C₉ cycloalkyl and —C₁-C₄alkyl(C₃-C₉cycloalkyl);
R² is H or an optionally substituted alkyl;
if R¹ is an optionally substituted C₃-C₉ cycloalkyl or an optionally substituted —C₁-C₄alkyl(C₃-C₉cycloalkyl), then any carbon of the R carbocyclic ring can be optionally substituted by both Y and Z;
Y and Z are independently selected from among halogen or methyl; or
Y and Z taken together form an oxo (═O);
one of A or B is -L-G and the other is selected from among H and an optionally substituted C₁-C₆ alkyl; or
A and B are both selected from among H and an optionally substituted C₁-C₆ alkyl provided that at least one X is present and is N;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is tetrazolyl, —NHS(═O)₂R⁸, —S(═O)₂NHR⁹, —OH, —SH, —N(R⁹)₂, —C(O)NHC(O)R⁸, —C(O)NHS(═O)₂R⁸, —S(═O)₂NHC(O)R⁸, —S(═O)₂NHC(O)NHR⁸, —C(═NR¹⁰)N(R⁹)₂, —NR⁹C(═NR¹⁰)N(R⁹)₂, —NR⁹C(═NR¹⁰)NHC(═NR¹⁰)N(R)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, a carboxylic acid bioisostere, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
X is CH or N, provided that at least one X is N;
n is 1, 2, 3, or 4; x is 1, 2, or 3; y is 0, 1, or 2; and pharmaceutically acceptable salts, pharmaceutically acceptable N-oxides, pharmaceutically active metabolites, pharmaceutically acceptable prodrugs, or pharmaceutically acceptable solvates thereof.

In some embodiments, compounds provided herein have a structure of Formula (I):

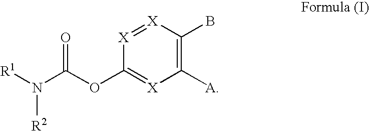

In some embodiments, compounds provided herein have a structure of Formula (Ia):

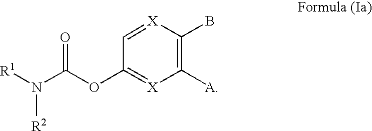

In other embodiments, compounds provided herein have a structure of Formula (Ib):

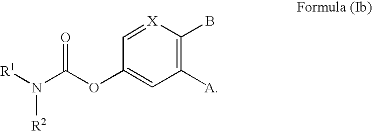

In some other embodiments, compounds provided herein have a structure of Formula (Ic):

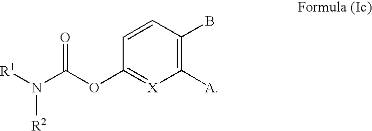

In another embodiment, compounds provided herein have a structure of Formula (Id):

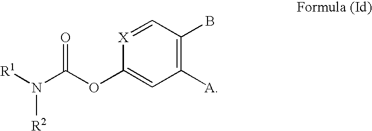

In yet some other embodiments, compounds provided herein have a structure of Formula (Ie):

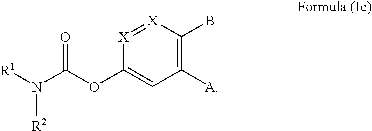

In yet some other embodiments, compounds provided herein have a structure of Formula (II):

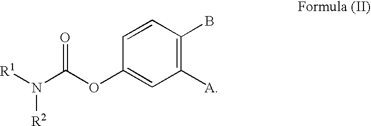

In some embodiments, both A and B are H provided that at least one X is present and is N. In embodiments where X is present, at least one X is N.

For any and all of the embodiments, if R¹ is an optionally substituted C₃-C₉ cycloalkyl or an optionally substituted —C₁-C₄alkyl(C₃-C₉cycloalkyl), then any carbon of the R¹ carbocyclic ring can be optionally substituted by both Y and Z; Y and Z are independently selected from among halogen or methyl; or Y and Z taken together form an oxo (═O).

For any and all of the embodiments, substituents can be selected from among from a subset of the listed alternatives. For example, in some embodiments, one of A or B is -L-G and the other is H or an optionally substituted C₁-C₆ alkyl. In other embodiments, one of A or B is -L-G and the other is H. In some embodiments, A is -L-G. In some other embodiments, B is -L-G. In some other embodiments, both A and B are selected from among H and an optionally substituted C₁-C₆ alkyl provided that at least one X is present and is N.

In certain embodiments, R² is H.

In some embodiments, L is a bond, or an optionally substituted group selected from among C₁-C₆ alkylene, C₁-C₆ketoalkylene, —C(O)NR⁹—(CH₂)ₙ—, —NR⁹—C(O)—(CH₂)ₙ—, or —S(O)₂—(CH₂)ₙ—.

In other embodiments, L is a bond. In some embodiments, if L is not a bond, L taken together with A or B can form a carbocyclic ring.

In certain embodiments, G is —N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, or —C(O)NR⁹C(═CHR¹⁰)N(R⁹)₂.

In some embodiments, each R¹⁰ is H. In certain embodiments, G is —N(R⁹)₂, —C(═NR¹⁰)N(R⁹)₂, or —NR⁹C(═NR¹⁰)N(R⁹)₂.

In other embodiments, G is tetrazolyl, —S(═O)₂NHR⁹, —OH, —SH, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)ₙOH; —OS(O)ₙOH; or —NR⁹S(O)_{y}OH. In some other embodiments, G is tetrazolyl, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₙOH, —OP(═O)OR⁸OH, —OP(═O)R⁸OH, —P(═O)OR⁸OH; —P(═O)R⁸OH, or —S(O)ₙOH. In yet other embodiments, G is tetrazolyl, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₙOH, or —S(O)_{y}OH; x is 1; and y is 1 or 2. In yet other embodiments, G is tetrazolyl, —CO₂H, or a carboxylic acid bioisostere.

In some embodiments, each R⁸ is independently a substituted or unsubstituted C₁-C₆ alkyl; each R⁹ is H; and each R¹⁰ is independently selected from among H, —S(═O)₂R⁸, and —C(O)R⁸. In some embodiments, each R⁹ is H. In some other embodiments, each R¹⁰ is H.

In some of the embodiments provided herein, R¹ is an optionally substituted group selected from among C₃-C₉cycloalkyl, and —C₁-C₄alkyl-(C₃-C₉cycloalkyl). In other embodiments, R¹ is an optionally substituted C₃-C₉cycloalkyl. In yet other embodiments, R¹ is an optionally substituted —C₁-C₄alkyl-(C₃-C₉cycloalkyl).

In some embodiments, R¹ is selected from among methyl, ethyl, propyl, isopropyl, n-butyl, sec-butyl, iso-butyl, tert-butyl, pentyl, neopentyl, hexyl, cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl. In certain embodiments, R¹ is selected from among isopropyl, sec-butyl, iso-butyl, tert-butyl, cyclopropyl, cyclobutyl, cyclopentyl, cyclohexyl, cycloheptyl, cyclopropylmethyl, cyclobutylmethyl, cyclopentylmethyl, cyclohexylmethyl and cycloheptylmethyl. In some of the embodiments provided herein, R¹ is selected from among isopropyl, sec-butyl, iso-butyl, tert-butyl, cyclopropyl, cyclopentyl, cyclohexyl, cyclopropylmethyl, cyclopentylmethyl, and cyclohexylmethyl. In some embodiments, R¹ is selected from among isopropyl, sec-butyl, iso-butyl, cyclohexyl, cyclopropylmethyl and cyclohexylmethyl. In certain embodiments, R¹ is selected from among cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl. In other embodiments, R¹ is selected from among cyclohexyl and cyclohexylmethyl.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some other embodiments, compounds provided herein have a structure of Formula (II):

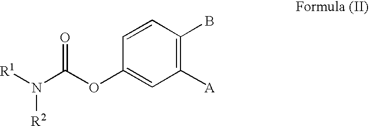

wherein:
R¹ is an optionally substituted group selected from among C₃-C₉ cycloalkyl and —C₁-C₄alkyl(C₃-C₉cycloalkyl);
R² is H or an optionally substituted alkyl;
if R¹ is an optionally substituted C₃-C₉ cycloalkyl or an optionally substituted —C₁-C₄alkyl(C₃-C₉cycloalkyl), then any carbon of the R¹ carbocyclic ring can be optionally substituted by both Y and Z;
Y and Z are independently selected from among halogen or methyl; or
Y and Z taken together form an oxo (═O); one of A or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —S(O)₂NH—(CH₂)ₙ—, —C(═NR¹⁰)N(R⁹)—(CH₂)ₙ—, and —NR⁹C(═NR¹⁰)N(R⁹)—(CH₂)ₙ—;
G is tetrazolyl, —NHS(═O)₂R⁸, —S(═O)₂NHR⁹, —OH, —SH, —N(R⁹)₂, —C(O)NHC(O)R⁸, —C(O)NHS(═O)₂R⁸, —S(═O)₂NHC(O)R⁸, —S(═O)₂NHC(O)NHR¹, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, a carboxylic acid bioisostere, —(OP(═O)OH)ₙOH, —OP(═O)OR¹⁰H, —OP(═O)R⁸OH, —NR⁹P(═O)OR⁸OH, —NR⁹P(═O)R⁸OH, —P(═O)OR⁸OH; —P(═O)R⁸OH, —S(O)ₙOH; —OS(O)_{y}OH; —NR⁹S(O)ₙOH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NH₂;
n is 1, 2, 3, or 4; x is 1, 2, or 3; y is 0, 1, or 2; and
pharmaceutically acceptable salts, pharmaceutically acceptable N-oxides, pharmaceutically active metabolites, pharmaceutically acceptable prodrugs, or pharmaceutically acceptable solvates thereof.

For any and all of the embodiments, substituents can be selected from among from a subset of the listed alternatives. For example, in some embodiments, one of A or B is -L-G and the other is H or an optionally substituted C₁-C₆ alkyl. In other embodiments, one of A or B is -L-G and the other is H. In some embodiments, A is -L-G. In some other embodiments, B is -L-G.

In certain embodiments, R² is H.

In some embodiments, L is a bond, or an optionally substituted group selected from among C₁-C₆ alkylene, C₁-C₆ketoalkylene, —C(O)NR⁹—(CH₂)ₙ—, —NR⁹—C(O)—(CH₂)ₙ—, —S(O)₂—(CH₂)ₙ—, and —S(O)₂NH—(CH₂)ₙ—. In some embodiments, L is a bond, or an optionally substituted group selected from among C₁-C₆ alkylene, and C₁-C₆ ketoalkylene. In other embodiments, L is a bond.

In certain embodiments, if L is not a bond, L taken together with A or B can form a carbocyclic ring.

In some embodiments, G is —N(R)₂, —C(═NR¹⁰)N(R⁹)₂, —NR⁹C(═NR¹⁰)N(R⁹)₂, —NR⁹C(═NR¹⁰)NHC(═NR¹⁰)N(R)₂, —NR⁹C(═CHR¹⁰)N(R⁹)₂, —C(O)NR⁹C(═NR¹⁰)N(R⁹)₂, or —C(O)NR⁹C(═CHR¹⁰)N(R⁹)₂. In some embodiments, G is —N(R⁹)₂, —C(═NR¹⁰)N(R)₂, or —NR⁹C(═NR¹⁰)N(R⁹)₂. In some other embodiments, each R¹⁰ is H.

In some embodiments, G is tetrazolyl, —NHS(═O)₂R¹, —S(═O)₂NHR⁹, —OH, —SH, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₓOH, —OP(═O)OR⁸OH, —OP(═O)R⁸H, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; or —NR⁹S(O)ₙOH. In other embodiments, G is tetrazolyl, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₙOH, —OP(═O)OR⁸OH, —OP(═O)R⁸OH, —P(═O)OR⁸OH; —P(═O)R⁸OH, or —S(O)_{y}OH. In some other embodiments, G is tetrazolyl, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₓOH, or —S(O)_{y}OH; x is 1; and y is 1 or 2. In yet some other embodiments, G is tetrazolyl, —CO₂H, or a carboxylic acid bioisostere.

In other embodiments, R¹ is an optionally substituted C₃-C₉cycloalkyl. In yet other embodiments, R¹ is an optionally substituted —C₁-C₄alkyl-(C₃-C₉cycloalkyl). In some embodiments, R¹ is selected from among cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl. In other embodiments, R¹ is selected from among cyclohexyl and cyclohexylmethyl.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments, provided herein is a compound selected from among: 3-(cyclohexylcarbamoyloxy)benzoic acid (Compound 1); 3-(cyclohexylmethylcarbamoyloxy)benzoic acid (Compound 2); 4-(cyclohexylcarbamoyloxy)benzoic acid (Compound 3); 4-(cyclohexylmethylcarbamoyloxy)benzoic acid (Compound 4); 2-(3-((cyclohexylmethyl)carbamoyl)phenyl)acetic acid (Compound 7); 5-(3-(cyclohexylcarbamoyl)phenyl)-5-oxopentanoic acid (Compound 8); 5-(3-((cyclohexylmethyl)carbamoyl)phenyl)-5-oxopentanoic acid (Compound 9); 3-(1H-tetrazol-5-yl)phenyl cyclohexylcarbamate (Compound 10); 3-(1H-tetrazol-5-yl)phenyl cyclohexylmethylcarbamate (Compound 11); 4-(3-(cyclohexylcarbamoyl)phenyl)-4-oxobutanoic acid (Compound 12); 4-(3-((cyclohexylmethyl)carbamoyl)phenyl)-4-oxobutanoic acid (Compound 13); and pharmaceutically acceptable salts and prodrugs thereof.

### Preparation of Compounds

Compounds provided herein that inhibit the activity of FAAH may be synthesized using standard synthetic techniques known to those of skill in the art or using methods known in the art in combination with methods described herein. As a further guide the following synthetic methods may also be utilized.

The reactions can be employed in a linear sequence to provide the compounds described herein or they may be used to synthesize fragments which are subsequently joined by the methods described herein and/or known in the art.

### Use of Protecting Groups

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates. In one embodiment, a compound containing both a carboxylic acid reactive moiety and a hydroxy reactive moiety may have one of the reactive moieties blocked while the other reactive moiety is not blocked.

Allyl blocking groups are useful in then presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

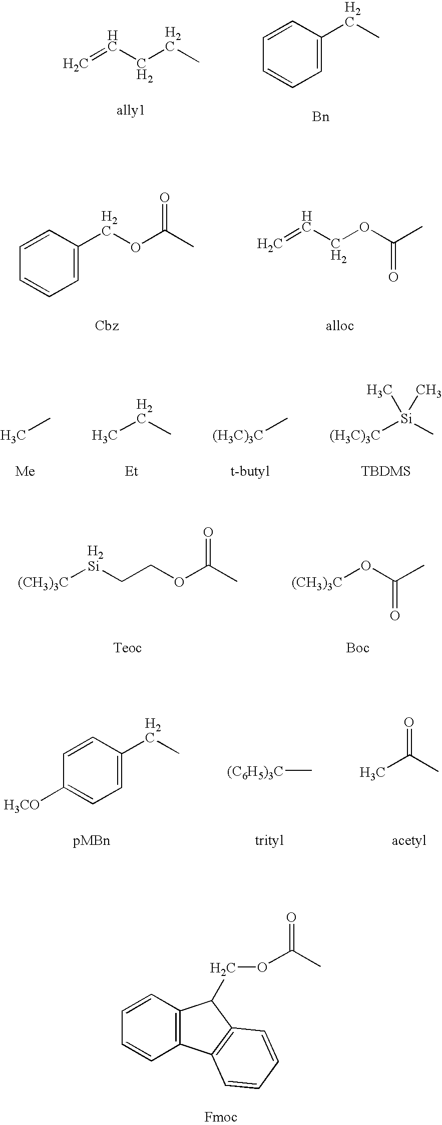

Other protecting groups are described in Greene and Wuts, Protective Groups in Organic Synthesis, 3rd Ed., John Wiley & Sons, New York, N.Y., 1999, which is incorporated herein by reference in its entirety.

### Process for the Preparation of Esters of Alkylcarbamic Acids

In certain embodiments, provided herein are methods of making and methods of using FAAH inhibitor compounds provided herein. In certain embodiments, compounds provided herein can be synthesized using the following synthetic schemes. Compounds may be synthesized using methodologies analogous to those described below by the use of appropriate alternative starting materials.

Described herein are compounds that inhibit the activity of fatty acid amide hydrolase (FAAH) and processes for their preparation. Also described herein are pharmaceutically acceptable salts, pharmaceutically acceptable N-oxides, pharmaceutically active metabolites and pharmaceutically acceptable prodrugs of such compounds. Pharmaceutical compositions that include at least one such compound or a pharmaceutically acceptable salt, pharmaceutically acceptable N-oxide, pharmaceutically active metabolite or pharmaceutically acceptable prodrug of such compound, are provided.

Esters of alkylcarbamic acids disclosed herein are prepared by the general process depicted in Scheme 1. Ar—OH (2) represents a hydroxy-containing compound selected from among substituted phenols and heteroaryls that include a hydroxyl moiety. R¹ and R² are as defined herein.

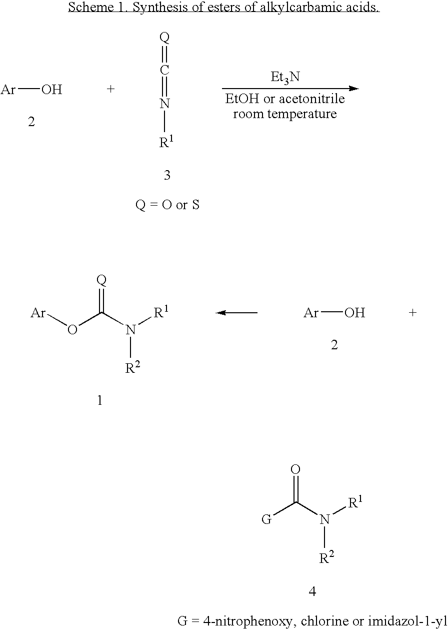

Treatment of Ar—OH (2) with an isocyanate or isothiocyanate (3) in the presence of a base, such as, for example, triethylamine, in an organic solvent, such as, for example, ethanol or acetonitrile, results in the formation of esters of alkylcarbamic acids of structure 1 (see, for example, U.S. Pat. No. 5,112,859; WO 2004/033422; US 2006/0014830; J. Med. Chem. 2004, 47(21); 4998-5008; Tarzia et al. J. Med. Chem. 46:2352-2360 (2003); Kathuria et al. Nature Medicine 9(1): 76 (2003)). Isocyanates or isothiocyanates are commercially available. Methods for the preparation of isocyanates or isothiocyanates (3) are well known in the art. For example, isocyanates (3, Q=O) can be prepared from the corresponding carboxylic acid (i.e. R¹—COOH) or acid derivative (e.g. R¹—C(O)Cl) by treatment with an azide source such as, for example, sodium azide or diphenylphosphoryl azide followed by a Curtius-type rearrangement (see, for example, Synth. Commun. 1993, 23, 335; Heterocycles 1993, 36, 1305).

Alternatively, alkylcarbamic acid esters (1) can be prepared by treatment of Ar—OH (2) with alkylcarbamic acid derivatives of structure (4), where G is 4-nitrophenoxy, chlorine or imidazol-1-yl, in the presence of a base, such as, for example, triethylamine, to provide the desired compound (1). Compounds of structure (4) can be prepared using procedures well known in the art, such as, procedures described in Greene, T. W. and Wuts, P. G. M “Protective Groups in Organic Synthesis”, 3rd Edition, p. 549, New York:Wiley, 1999. Briefly, alkylamines (e.g. R₁—NH₂) are treated with phosgene or a phosgene equivalent, such as, for example, trichloromethyl chloroformate or carbonyldiimidazole, to yield compounds of structure (4).

Esters of alkyl(thio)carbamic acids also can be synthesized by the method outlined in Scheme 2.

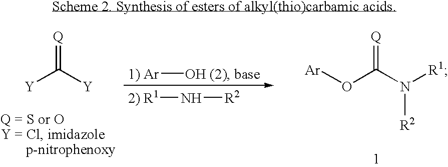

Esters of alkyl(thio)carbamic acids can be prepared by a two-step procedure. Thiophosgene, phosgene, or an equivalent thereof, is first treated with Ar—OH (2) in the presence of a base in a suitable organic solvent, followed by treatment with an alkylamine such as, R¹R²NH. The order of the reaction can be reversed, i.e. thiophosgene, phosgene, or an equivalent thereof, can be treated with the alkylamine followed by Ar—OH (2). Equivalents of thiophosgene and phosgene include, but are not limited to, 1,1′-thiocarbonyldiimidazole, 1,1′-carbonyldimidazole, and trichloromethyl chloroformate.

The requisite hydroxy-containing compounds, Ar—OH (2), can be purchased from commercial sources or prepared using procedures known in the art or outlined herein.

Using the reaction conditions described herein, esters of alkylcarbamic acids as disclosed herein are obtained in good yields and purity. The compounds prepared by the methods disclosed herein are purified by conventional means known in the art, such as, for example, filtration, recrystallization, chromatography, distillation, and combinations thereof.

Any combination of the groups described above for the various variables is contemplated herein.

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

Provided herein are pharmaceutical compositions that include a compound described herein and a pharmaceutically acceptable diluent(s), excipient(s), or carrier(s). In addition, the compounds described herein can be administered as pharmaceutical compositions in which compounds described herein are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

A pharmaceutical composition, as used herein, refers to a mixture of a compound described herein with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors.

The compounds can be used singly or in combination with one or more therapeutic agents as components of mixtures.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

### Certain Pharmaceutical Terminology

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “acceptable” with respect to a formulation, composition or ingredient, as used herein, means having no persistent detrimental effect on the general health of the subject being treated.

As used herein, the term “selective inhibitor compound” refers to a compound that selectively inhibits a specific function/activity of one or more target proteins.

As used herein, the term “selectively inhibits” refers to the ability of a selective inhibitor compound to inhibit a specific function/activity of a target protein, such as, for example, the fatty acid amide hydrolytic activity of fatty acid amide hydrolase, with greater potency than the activity of a non-target protein. In certain embodiments, selectively inhibiting refers to inhibiting a target protein activity with a selective inhibitor that has a IC₅₀ that is at least 10, 50, 100, 250, 500, 1000 or more times lower than for that of a non-target protein activity.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “selective modulator” refers to a compound that selectively modulates a target activity.

As used herein, the term “selective FAAH modulator” refers to a compound that selectively modulates at least one activity associated with FAAH.

As used herein, the term “selectively modulates” refers to the ability of a selective modulator to modulate a target activity to a greater extent than it modulates a non-target activity. In certain embodiments the target activity is selectively modulated by, for example about 2 fold up to more that about 500 fold, in some embodiments, about 2, 5, 10, 50, 100, 150, 200, 250, 300, 350, 400, 450 or more than 500 fold.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the IC₅₀ refers to an amount, concentration or dosage of a particular test compound that achieves a 50% inhibition of a maximal response, such as inhibition of FAAH, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, “physiological pH” refers to the pH of biological tissues. In one embodiment, physiological pH refers to the pH of blood. In another embodiment, physiological pH refers to the pH of plasma. In some embodiments, physiological pH refers to a pH of about 6.0 to about 7.4. In other embodiments, physiological pH refers to a pH of about 6.8 to about 7.4.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound administered, age, weight, general condition of the subject, the condition being treated, the severity of the condition being treated, and the judgment of the prescribing physician.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401).

By “pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. Pharmaceutically acceptable salts may be obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts also may be obtained by reacting a compound described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 10% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, 0) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

“Bioavailability” refers to the percentage of the weight of compounds disclosed herein dosed that is delivered into the general circulation of the animal or human being studied. The total exposure (AUC_{(0-∞)} of a drug when administered intravenously is usually defined as 100% bioavailable (F %). “Oral bioavailability” refers to the extent to which compounds disclosed herein are absorbed into the general circulation when the pharmaceutical composition is taken orally as compared to intravenous injection.

“Blood plasma concentration” refers to the concentration of compounds provided herein in the plasma component of blood of a subject. It is understood that the plasma concentration of compounds provided herein may vary significantly between subjects, due to variability with respect to metabolism and/or possible interactions with other therapeutic agents. In accordance with one embodiment disclosed herein, the blood plasma concentration of the compounds provided herein may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound provided herein may vary from subject to subject.

“Carrier materials” include any commonly used excipients in pharmaceutics and should be selected on the basis of compatibility with compounds disclosed herein and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F680®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per ml, dl, or 1 of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

Moreover, the pharmaceutical compositions described herein, which include a compound provided herein, can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of a compound provided herein, with one or more pharmaceutical excipients to form a bulk blend composition. When referring to these bulk blend compositions as homogeneous, it is meant that the particles of the compound provided herein, are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound provided herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of the compound provided herein. In one embodiment, some or all of the particles of the compound provided herein are coated. In another embodiment, some or all of the particles of the compound provided herein are microencapsulated. In still another embodiment, the particles of the compound provided herein are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound disclosed herein from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH1101, Avicel® PH1102, Avicel® PH 105, Elcema® P 100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the present invention. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of the compound disclosed herein from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the compound disclosed herein and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with compounds disclosed herein, which sufficiently isolate the compound disclosed herein from other non-compatible excipients. Materials compatible with compounds disclosed herein are those that delay the release of the compounds disclosed herein in vivo.

Exemplary microencapsulation materials useful for delaying the release of the formulations including compounds disclosed herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds disclosed herein may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of compounds disclosed herein are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds disclosed herein are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound disclosed herein may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In other embodiments, the formulations described herein, which include a compound disclosed herein, are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734, each of which is specifically incorporated by reference. In still other embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518, each of which is specifically incorporated by reference.

The pharmaceutical solid oral dosage forms including formulations described herein, which include a compound disclosed herein, can be further formulated to provide a controlled release of the compound disclosed herein. Controlled release refers to the release of the compound disclosed herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. In some embodiments the polymers for use in the present invention are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include a compound disclosed herein, are delivered using a pulsatile dosage form. A pulsatile dosage form is capable of providing one or more immediate release pulses at predetermined time points after a controlled lag time or at specific sites. Pulsatile dosage forms including the formulations described herein, which include a compound disclosed herein, may be administered using a variety of pulsatile formulations known in the art. For example, such formulations include, but are not limited to, those described in U.S. Pat. Nos. 5,011,692, 5,017,381, 5,229,135, and 5,840,329, each of which is specifically incorporated by reference. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284, all of which are specifically incorporated by reference. In one embodiment, the controlled release dosage form is pulsatile release solid oral dosage form including at least two groups of particles, (i.e. multiparticulate) each containing the formulation described herein. The first group of particles provides a substantially immediate dose of the compound disclosed herein upon ingestion by a mammal. The first group of particles can be either uncoated or include a coating and/or sealant. The second group of particles includes coated particles, which includes from about 2% to about 75%, preferably from about 2.5% to about 70%, and more preferably from about 40% to about 70%, by weight of the total dose of the compound disclosed herein in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD 100, Eudragit® E10, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the formulation that includes a compound disclosed herein.

Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference.

In some embodiments, pharmaceutical formulations are provided that include particles of the compounds disclosed herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition to the particles of compound disclosed herein, the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel®PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated by reference. Formulations that include a compound provided herein, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds disclosed herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations that include compounds disclosed herein may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the compound disclosed herein is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the compound disclosed herein, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In one embodiments, the transdermal formulations described herein include at least three components: (1) a formulation of a compound disclosed herein; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compounds disclosed herein. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include a compound disclosed herein, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Other Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Methods of Dosing and Treatment Regimens

The compounds described herein can be used in the preparation of medicaments for the inhibition of fatty acid amide hydrolase, or for the treatment of diseases or conditions that would benefit, at least in part, from inhibition of fatty acid amide hydrolase. In addition, a method for treating any of the diseases or conditions described herein in a subject in need of such treatment, involves administration of pharmaceutical compositions containing at least one compound disclosed herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The daily dosages appropriate for the compounds described herein described herein are from about 0.01 to 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Treatments

The compositions and methods described herein may also be used in conjunction with other well known therapeutic reagents that are selected for their particular usefulness against the condition that is being treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In certain instances, it may be appropriate to administer at least one compound described herein in combination with another therapeutic agent. By way of example only, if one of the side effects experienced by a patient upon receiving one of the compounds herein is nausea, then it may be appropriate to administer an anti-nausea agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

The compounds described herein and combination therapies can be administered before, during or after the occurrence of a disease or condition, and the timing of administering the composition containing a compound can vary. Thus, for example, the compounds can be used as a prophylactic and can be administered continuously to subjects with a propensity to develop conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound is preferably administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, preferably about 1 month to about 5 years, and more preferably from about 1 month to about 3 years.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of fatty acid amide hydrolase (FAAH), or in which FAAH is a mediator or contributor to the symptoms or cause.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically may include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### Treatments and/or Uses of FAAH Inhibitor Compounds

The enzyme FAAH catalyzes the hydrolysis of endogenous amide and ester derivatives of various fatty acids such as, but not limited to, N-arachidonoylethanolamine (anandamide), N-palmitoylethanolamine, N-oleoylethanolamine, oleamide and 2-arachidonoylglycerol. These derivatives exert different pharmacological activities by interacting, inter alia, with the cannabinoid and vanilloid receptors. Compounds provided herein block this degradation pathway and increase the tissue content of these endogenous substances. They may be used in this respect in the prevention and treatment of pathologies in which the endogenous cannabinoids, and/or any other substrates metabolized by the enzyme FAAH, are involved.

In some embodiments, compounds provided herein can be used to treat and/or prevent emesis, dizziness, vomiting, and nausea, especially after chemotherapy.

In some embodiments, compounds provided herein may be administered to alleviate pain in a subject. The treatment may be prophylactic or therapeutic. The treatment may be administered to a human subject. The treatment may or may not be administered in a combination therapy with another pain reliever or anti-inflammatory agent.

In some embodiments, compounds provided herein can be used in treatment of all varieties of pain including pain associated with a cough condition, pain associated with cancer, preoperative pain, arthritic pain and other forms of chronic pain such as post-operative pain, lumbosacral pain, musculo-skeletal pain, headache, migraine, muscle ache, lower back and neck pain, toothache and the like. In some embodiments, the compounds provided herein are useful for the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; neuralgia, such as post-herpetic neuralgia and trigeminal neuralgia; and pain resulting from physical trauma, amputation, cancer, toxins or chronic inflammatory conditions. The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

In some embodiments, compounds provided herein are useful in the prevention and/or treatment of pain, in particular acute or chronic neurogenic pain, migraine, neuropathic pains including the forms associated with herpes virus and diabetes, acute or chronic pain associated with the inflammatory diseases: arthritis, rheumatoid arthritis, osteoarthritis, spondylitis, gout, vascularitis, Crohn's disease, irritable bowel syndrome and acute/sharp or chronic pains at the periphery.

In some embodiments, compounds and compositions provided herein, may be used to treat non-inflammatory pain and/or inflammatory pain. The compounds are administered as therapeutics for various types of non-inflammatory pain including, without limitation:
peripheral neuropathic pain, which is pain caused by a lesion or dysfunction in the peripheral nervous system, for example, painful neuropathies where pain persists long after the tissue damage has healed;
central pain, which is pain caused by a lesion or dysfunction of the central nervous system, for example, thalmic lesions accompanied by severe pain in an unaffected part of the body;
deafferentation pain, which is pain due to loss of sensory impart into the central nervous system, for example, pain resulting from an injury where dorsal roots are torn away from the spinal cord;
chronic nociceptive pain, for example, certain types of cancer pain;
noxious stimulus of nociceptive receptors, such as, for example, pain felt in response to tissue damage or impending tissue damage;
phantom pain, which is pain felt in a part of the body that no longer exists;
pain felt by psychiatric patients, which is pain where no physical cause may exist;
wandering pain, wherein the pain repeatedly changes location in the body.

Compounds provided herein may be used to treat both non-inflammatory pain and inflammatory pain.

In another embodiment, compositions provided herein include a compound provided herein and at least one art-recognized analgesic or anti-inflammatory substance that is compatible with the compounds provided herein and preferably effective in oral dosage form. Examples of art-recognized analgesics and anti-inflammatory compounds include, but are not limited to: aspirin, carbaspirin, choline salicylate, diflunisal, magnesium salicylate, salicylamide, salicylic acid, salsalate, sodium thiosalicylate, acetaminophen, phenacetin, aminopyrine, mefenamic acid, methotrimeprazine, oxyphenbutazone, phenylbutazone, indomethacin, ibuprofen, sulindac, piroxicam, meclofenamate, zomepirac, codeine, morphine, meperidine, pethinine, alphaprodine, fentanyl, levorphanol, methadone, phenazocine, butorphanol, ethobeptozine, nalbuphine, pentazocine, propoxyphene, fenoprofen, naproxen, tolmeton and the like. In a preferred embodiment, a compound provided herein is co-administered with such an art-recognized analgesic and/or anti-inflammatory compound resulting in a synergistic anti-inflammatory and/or analgesic effect.

In another embodiment, compounds and compositions provided herein may be used for reducing neuropathic pain.

As used herein, the term “neuropathic pain” means pain resulting from injury to a nerve. Neuropathic pain is distinguished from nociceptive pain, which is the pain caused by acute tissue injury involving small cutaneous nerves or small nerves in muscle or connective tissue. Pain involving a nociceptive mechanism usually is limited in duration to the period of tissue repair and generally is alleviated by available analgesic agents or opioids (Myers, Regional Anesthesia 20:173-184 (1995), which is incorporated herein by reference).

Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Compounds provided herein may be used in alleviating neuropathic pain regardless of the etiology of the pain. In certain embodiments, compounds and compositions provided herein can be used to alleviate neuropathic pain resulting from a peripheral nerve disorder such as neuroma; nerve compression; nerve crush, nerve stretch or incomplete nerve transsection; mononeuropathy or polyneuropathy.

A neuroma can develop readily after traumatic injury to nerve, especially when a whole nerve is severely crushed or transsected. In a neuroma, the neurite outgrowth that normally regenerates a peripheral nerve is aberrant or misguided due, for-example, to a physical obstruction such as scar tissue. Thus, a regenerating nerve fiber is entangled in an environment in which mechanical and physical factors precipitate abnormal electrophysiologic activity and pain (Myers, supra, 1995). An amputation neuroma, for example, can cause phantom pain or can cause pain triggered by the use of a limb prosthesis. As disclosed herein, such neuropathic pain can be alleviated by administration of a FAAH inhibitor, such as, for example, a compound provided herein.

Nerve compression also results in neuropathic pain. Nerve compression can be abrupt, as in the case of traumatic nerve crush, or can be prolonged and moderate, secondary to tumor growth or scar formation in the proximity of a major nerve bundle. Compression neuropathy can occur as a result of changes in blood flow to a nerve, causing severe ischemia and consequent nerve injury (Myers, supra, 1995).

In other embodiments, compounds and compositions provided herein can be used to alleviate neuropathic pain resulting from a disorder such as dorsal root ganglion compression; inflammation of the spinal cord; contusion, tumor or hemisection of the spinal cord; tumors of the brainstem, thalamus or cortex; or trauma to the brainstem, thalamus or cortex.

In some embodiments, administration of a compound provided herein can alleviate neuropathic pain resulting from a mononeuropathy or polyneuropathy. As used herein, a neuropathy is a functional disturbance or pathological change in the peripheral nervous system and is characterized clinically by sensory or motor neuron abnormalities. The term mononeuropathy indicates that a single peripheral nerve is affected, while the term polyneuropathy indicates that several peripheral nerves are affected. The etiology of a neuropathy can be known or unknown (see, for example, Myers, supra, 1995; Galer, Neurology 45(suppl 9):S17-S25 (1995); Stevens and Lowe, Pathology, Times Mirror International Publishers Limited, London (1995)). Known etiologies include complications of a disease or toxic state; for example, diabetes is the most common metabolic disorder causing neuropathy. In some embodiments, a compound provided herein alleviates the neuropathic pain of a mononeuropathy resulting, for example, from diabetes, irradiation, ischemia or vasculitis. In other embodiments, a compound provided herein alleviates the neuropathic pain of a polyneuropathy resulting, for example, from post-polio syndrome, diabetes, alcohol, amyloid, toxins, HIV, hypothyroidism, uremia, vitamin deficiencies, chemotherapy, ddC or Fabry's disease. In some other embodiments, compounds provided herein also can alleviate neuropathic pain of unknown etiology.

In some embodiments, compounds provided herein may be used to treat inflammatory disorders, such as, for example, auto-immune disorders.

The term “inflammatory disorders” refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which may be partial or complete, temporary or permanent). Inflammation takes many forms and includes, but is not limited to, inflammation that is one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporal arteritis); joints (arthritis: crystalline, osteo-, psoriatic, reactive, rheumatoid, Reiter's); gastrointestinal tract (chron's disease, ulcerative colitis); skin (dermatitis); or multiple organs and tissues (systemic lupus erythematosus) [Harrison's Principles of Internal Medicine, 16th Edition, Kasper D L, et al, Editors; McGraw-Hill, publishers].

Immune disorders, such as auto-immune disorders, which can be treated with a compound provided herein or compositions that include a compound provided herein include: arthritis (including rheumatoid arthritis, spondyloarthopathies, gouty arthritis, degenerative joint diseases (i.e. osteoarthritis), systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin inflammation disorders (i.e. psoriasis, eczema, burns, dermatitis), enuresis, eosinophilic disease, gastrointestinal disorders (including inflammatory bowel disease, peptic ulcers, regional enteritis, diverticulitis, gastrointestinal bleeding, Crohn's disease, gastritis, diarrhoea, irritable bowel syndrome and ulcerative colitis), and disorders ameliorated by a gastroprokinetic agent (i.e. ileus, for example postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP)).

Compositions that include a compound provided herein can also be used to treat, for example, inflammation associated with: vascular diseases, migraine headaches, tension headaches, periarteritis nodosa, thyroiditis, aplastic anemia, Hodgkin's disease, scierodoma, rheumatic fever, type I diabetes, myasthenia gravis, sarcoidosis, nephrotic syndrome, Behcet's syndrome, polymyositis, gingivitis, hypersensitivity, conjunctivitis, multiple sclerosis, and ischemia (e.g., myocardial ischemia), and the like. The compounds may be useful for treating neuroinflammation associated with brain disorders (e.g., Parkinson's disease and Alzheimer's disease) and chronic inflammation associated with cranial radiation injury. The compounds may be useful for treating acute inflammatory conditions (such as those resulting from infection) and chronic inflammatory conditions (such as those resulting from asthma, arthritis and inflammatory bowel disease). The compounds may also be useful in treating inflammation associated with trauma and non-inflammatory myalgia. The compounds can also be administered to those prior to surgery or taking anticoagulants. The compounds provided herein may reduce the risk of a thrombotic cardiovascular event which is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fagax), reversible ischemic neurologic deficits, and any similar thrombotic event in any vascular bed (splanchnic, renal, aortic, peripheral, etc.).

The compounds provided herein can be used in the treatment of symptoms associated with influenza or other viral infections, common cold, sprains and strains, myositis, neuralgia, synovitis, injuries such as sports injuries and those following surgical and dental procedures, coagulation disorders, kidney disease (e.g., impaired renal function), ophthalmic disorders (including glaucoma, retinitis, retinopathies, uveitis, wet macular degeneration, and acute injury to the eye tissue), liver diseases (i.e., inflammatory liver disease including chronic viral hepatitis B, chronic viral hepatitis C, alcoholic liver injury, primary biliary cirrhosis, autoimmune hepatitis, nonalcoholic steatohepatitis and liver transplant rejection), and pulmonary inflammatory diseases (e.g., including asthma, allergic rhinitis, respiratory distress syndrome chronic bronchitis, and emphysema).

The compounds provided herein may be used to inhibit uterus contraction caused by hormones and prostanoid-induced smooth muscle contraction. The compounds provided herein may be useful in treating premature labor, menstrual cramps, menstrual irregularity, and dysmenorrhea.

In some embodiments, the compounds provided herein may inhibit cellular neoplastic transformations and metastatic tumor growth. The compounds may be associated with reducing the number of adenomatous colorectal polyps. Thus, compounds provided herein may be useful in reducing the risk of certain cancers, e.g., solid tumor cancers such as colon or colorectal cancer. In certain embodiments, compounds provided herein may be used in the treatment or prevention of cancer, such as, but not limited to, cancers of the bladder, cancers associated with overexpression of HER-2/neu cervix, skin, esophagus, head and neck, lung including non small-cell lung cancers, kidney, pancreas, prostate, gall bladder and bile duct and endometrial cancers, gastric cancers, gliomas, hepatocellular carcinomas, colonic adenomas, mammary cancers, ovarian cancers and salivary cancers. In addition, the compounds provided herein may be used in treating large intestine cancer and prostate cancer. The compounds may also be used in cases where the patient is at risk for cancer including oral premalignant lesions, cervical intraepithelial neoplasia, chronic hepatitis, bile duct hyperplasia, atypical adenomatous hyperplasia of lung, prostatic, intraepithelial neoplasia, bladder dysplasia, actinic keratoses of skin, colorectal adenomas, gastric metaplasia, and Barrett's esophagus.

In certain embodiments, compounds provided herein may be used to treat cancers, such as, but not limited to, benign tumors of the skin, papillomas and cerebral tumors, prostate tumors, cerebral tumors, glioblasiomas, medullary epitheliomas, medullary blastomas, neuroblastomas, tumors of embryonic origin, astrocytomas, astroblastomas, ependymomas, oligodendrogliomas, plexus tumor, neuroepithelioma, epiphysis tumor, ependyblastomas, malignant meningiomas, sarcomatosis, malignant melanomas, and schwennomas.

In some embodiments, compounds provided herein may also be of use in the treatment and/or prevention of cyclooxygenase-mediated proliferative disorders such as may occur in diabetic retinopathy and tumor angiogenesis. In some embodiments, compounds provided herein may be used to inhibit angiogenesis, such as occurs in wet macular degeneration.

FAAH inhibitors, such as, for example, compounds provided herein, are useful for the treatment of cognitive disorders such as dementia, particularly degenerative dementia (including senile dementia, Alzheimer's disease (and precursors thereof), Pick's disease, Huntington's chorea, Parkinson's disease and Creutzfeldt-Jakob disease), and vascular dementia (including multiinfarct dementia), as well as dementia associated with intracranial space occupying lesions, trauma, infections and related conditions (including HIV infection), metabolism, toxins, anoxia and vitamin deficiency; and mild cognitive impairment associated with ageing, particularly Age Associated Memory Impairment.

FAAH inhibitors, such as, for example, compounds provided herein, may prevent neuronal injury by inhibiting the generation of neuronal free radicals (and hence oxidative stress) and therefore are of use in the treatment of stroke; epilepsy; and epileptic seizures (including grand mal, petit mal, myocloñic epilepsy and partial seizures). FAAH inhibitors, such as, compounds provided herein, may be useful to control or suppress seizures (including those that are chemically induced).

In some embodiments, FAAH inhibitor compounds, such as, for example, compounds provided herein, may be used in acute and chronic neurodegenerative diseases, such as, for example, Parkinson's disease, Alzheimer's disease, senile dementia, Huntington's chorea, lesions associated with cerebral ischaemia and cranial and medullary trauma.

In some embodiments, FAAH inhibitory compounds, such as, for example, compounds provided herein, and compositions that include them, may be useful in treating depression and depressive disorders or conditions. The compounds and compositions may be useful, for example in treating major depressive disorders (unipolar depression), dysthymic disorders (chronic, mild depression), and bipolar disorders (manic-depression). The depression may be clinical or subclinical depression.

FAAH inhibitory compounds, such as, for example, compounds provided herein, and compositions that include the compounds, may be used in treating anxiety and anxiety disorders or conditions. These compounds and compositions are useful, for example in treating anxiety, clinical anxiety, panic disorder, agoraphobia, generalized anxiety disorder, specific phobia, social phobia, obsessive-compulsive disorder, acute stress disorder, and post-traumatic stress disorder; and adjustment disorders with anxious features, anxiety disorders due to general medical conditions, substance-induced anxiety disorders, and the residual category of anxiety disorder not otherwise specified. The treatment may be prophylactic or therapeutic. The compounds may be used for treating anxiety and anxiety disorders or conditions alone and/or also may be useful for concurrently treating another disorder or condition, such as, for example, pain, obesity, depression, or other disorder.

In some embodiments, a compound that inhibits the activity of FAAH, such as for example, compounds provided herein, and compositions that include the compounds, are useful in treating epilepsy and convulsive disorders or seizures. The compounds and compositions of the invention may be administered solely for the purposes of reducing the severity or frequency of convulsions or seizures.

In some embodiments, inhibition of FAAH induces sleep (U.S. Pat. Nos. 6,096,784; 6,096,784; 6,271,015; WO 98/24396). In one embodiment, the compounds provided herein can be administered to a mammal and the subsequent time (e.g., onset, duration) spent sleeping (e.g., eyes closed, motor quiescence) can be increased.

In some embodiments, a FAAH inhibitor compound may be useful in treating schizophrenia and dopamine related disorders. In some embodiments, the compounds and compositions provided herein are useful in treating schizophrenia, paranoia, paranoid ideation, flat affect or other related disorders, or other disorders of dopamine transmission.

In some embodiments, the compounds provided herein and compositions that include the compounds provided herein may be administered to induce or promote sleep in a mammal. The treatment may be prophylactic or therapeutic and may be administered to a healthy human patient solely for the purposes of reducing the severity or frequency or extent of sleeplessness. In other embodiments, compounds and compositions provided herein may be used to treat sleeping disorders, such as, but not limited to, insomnia and sleep apnoea.

In some embodiments, compounds that inhibit the activity of FAAH, such as, for example, compounds provided herein, and compositions that include the compounds, may be used to reduce appetite(s), reduce body fat and for treating or preventing obesity or overweight in a mammal and for preventing or treating the diseases associated with these health conditions. In one embodiment, administration of a FAAH inhibitor, such as, for example, a compound provided herein, may be used in reducing appetite, body fat or body weight, or for treating or preventing obesity or overweight, or for reducing food intake, or treating an appetency disorder in a mammal.

In one embodiment, compounds and compositions provided herein may be used for reducing appetite, body fat or body weight, or for treating or preventing obesity or overweight, or for reducing food intake, or treating an appetency disorder in a human patient, including alteration of body mass composition such as percent fat or alteration in lean muscle mass.

In a further embodiment, the FAAH inhibitor, such as, for example, compound provided herein, is administered in a combination therapy with oleoylethanolamide (OEA) or another fatty acid alkanolamide compound, homologue or analog, which a) reduces appetite, reduces food consumption, reduces body fat or reduces body weight and b) is subject to hydrolysis by FAAH.

In some embodiments, a FAAH inhibitor, such as, for example, compound provided herein, is administered to a subject in amounts sufficient to reduce body fat, body weight, or prevent body fat or body weight gain or to reduce appetite(s). In another embodiment, compositions provided herein include a FAAH inhibitor, such as, for example, a compound provided herein, and oleoylethanolamide, or a fatty acid amide compound, homologue or analog thereof.

In certain embodiments, FAAH inhibitors, such as, compounds provided herein, can be used to treat various metabolic disorders such as insulin resistance, diabetes, steatohepatitis, hyperlipidemia, fatty liver disease, non-alcoholic steatohepatitis, atherosclerosis and arteriosclerosis. Methods for measuring the affect of the compounds provided herein on such disorders are disclosed in U.S. Pat. No. 6,946,491, which is incorporated herein by reference.

In one embodiment, compounds provided herein and pharmaceutical compositions that include the compounds provided herein, may be used to treat a condition selected from among insulin resistance syndrome and diabetes (both primary essential diabetes such as Type I Diabetes or Type II Diabetes and secondary nonessential diabetes). Administration of a compound or composition provided herein can reduce a symptom of diabetes or the chance of developing a symptom of diabetes, such as atherosclerosis, hypertension, hyperlipidemia, fatty liver disease, nephropathy, neuropathy, retinopathy, foot ulceration and cataracts, each such symptom being associated with diabetes.

In one embodiment, compounds provided herein, and pharmaceutical compositions that include the compounds provided herein, can be used to treat hyperlipidemia. Administration of a compound or composition provided herein can reduce serum triglycerides and free fatty acids in hyperlipidemic subjects. In one embodiment, compounds and pharmaceutical compositions provided herein can be used to treat fatty liver disease. In another embodiment, compounds and pharmaceutical compositions provided herein can be used to treat atherosclerosis or arteriosclerosis.

In other embodiments, compounds and compositions provided herein may be used to treat and/or prevent food behavioral problems/feeding disorders (i.e. eating disorders, in particular anorexias and cachexias of various natures, weight loss associated with cancer and other wasting conditions).

In some embodiments, FAAH inhibitors, such as compounds provided herein, may be administered to treat or prevent glaucoma or to reduce intraocular eye pressure. In some embodiments, the compounds may be given systemically. In other embodiments, the FAAH inhibitors are direct applied to the surface of the eye (e.g., via eye drops). Ocular carrier formulations for such ocular application are taught in Remington's Pharmaceutical Sciences, Gennaro A R ed. 20th edition, 2000: Williams & Wilkins Pa., USA.

In some embodiments, compounds provided herein, and compositions that include the compounds can be administered to treat or prevent glaucoma or to reduce intraocular eye pressure. In some embodiments, the compounds may be given systemically. In other embodiments, the compounds are direct applied to the surface of the eye (e.g., via eye drops).

Other diseases, disorders, and/or conditions that can be treated and/or prevented with a compound provided herein, include: lung diseases (i.e. diseases of the respiratory tracts, bronchyospasms, cough, asthma, chronic bronchitis, chronic obstruction of the respiratory tracts, emphysema); urinary incontinence, inflammation of the bladder, urinary incontinence, vesical inflammation, movement disorders, psychomotor disorders, hypertension; cardiovascular diseases, in particular hypertension, cardiac arrhythmia, arteriosclerosis, heart attack, cardiac ischaemia, renal ischaemia; neurological pathologies, psychiatric tremors, dyskinesias, dystonia, spasticity, obsessive compulsive behavior, Tourette's syndrome, mood disturbances, psychoses.

Any combination of the disorders, diseases and/or conditions listed herein may be treated with the compounds provided herein.

### EXAMPLES

The person skilled in the art may further appreciate various aspects and advantages of the present disclosure upon review of the following illustrative and non-limiting examples:

### General Procedure for the Preparation of Esters of Alkylcarbamic Acids

To a stirred suspension or solution of a hydroxy-containing compound, in ethanol or acetonitrile, was added triethylamine and an alkylisocyanate (or alkylisothiocyanate). The reaction was stirred at room temperature for 2-16 hours. An additional amount of the alkylisocyanate (or alkylisothiocyanate) is added if needed. The reaction mixture is quenched with 10% HCl (aqueous solution) and the product is filtered (if precipitation occurs); if precipitation does not occur, the product is extracted with ethyl acetate and purified by chromatography. Otherwise the reaction mixture is purified by methods known in the art, such as, but not limited to, chromatography, filtration, distillation, recrystallization, and combinations thereof.

### Example 1

### 3-(Cyclohexylcarbamoyloxy)benzoic acid (Compound 1)

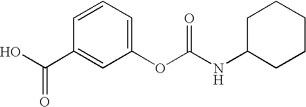

To a solution of 3-hydroxy benzoic acid (0.75 mmol) in acetonitrile (3 mL) was added cyclohexyl isocyanate (0.75 mmol, 0.11 mL) and triethylamine (0.75 mmol, 0.12 mL). The reaction mixture was stirred at room temperature for 12-16 hours, and then treated with 10% aqueous HCl. The reaction mixture was then purified by reversed-phase HPLC to yield the product as a white solid. MS (ESI) MH+: 264.

### Example 2

### 3-(Cyclohexylmethylcarbamoyloxy)benzoic acid (Compound 2)

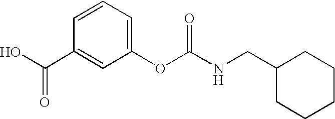

3-(Cyclohexylmethylcarbamoyloxy)benzoic acid was prepared from cyclohexylmethyl isocyanate and 3-hydroxy benzoic acid following the procedure outlined in Example 1. MS (ESI) MH+: 278.

### Example 3

### 4-(Cyclohexylcarbamoyloxy)benzoic acid (Compound 3)

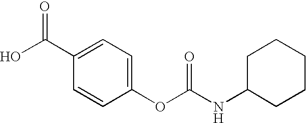

4-(Cyclohexylcarbamoyloxy)benzoic acid was prepared from cyclohexyl isocyanate and 4-hydroxy benzoic acid following the procedure outlined in Example 1. MS (ESI) MH+: 264.

### Example 4

### 4-(Cyclohexylmethylcarbamoyloxy)benzoic acid (Compound 4)

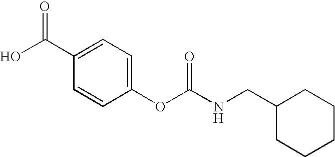

4-(Cyclohexylcarbamoyloxy)benzoic acid was prepared from cyclohexylmethyl isocyanate and 4-hydroxybenzoic acid following the procedure outlined in Example 1. MS (ESI) MH+: 278.

### Example 5

### 2-(3-((Cyclohexylmethyl)carbamoyl)phenyl)acetic acid (Compound 7)

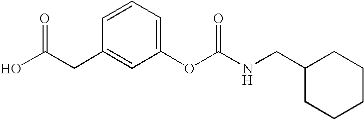

2-(3-((Cyclohexylmethyl)carbamoyl)phenyl)acetic acid was prepared from cyclohexylmethylisocyanate and 3-hydroxyphenylacetic acid following the procedure outlined in Example 1.

### Example 6

### 5-(3-(Cyclohexylcarbamoyl)phenyl)-5-oxopentanoic acid (Compound 8)

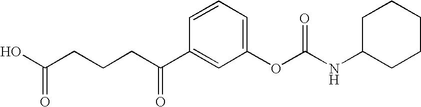

5-(3-(Cyclohexylcarbamoyl)phenyl)-5-oxopentanoic acid was prepared from cyclohexylisocyanate and 5-(3-hydroxyphenyl)-5-oxopentanoic acid following the procedure outlined in Example 1.

### Example 7

### 5-(3-((Cyclohexylmethyl)carbamoyl)phenyl)-5-oxopentanoic acid (Compound 9)

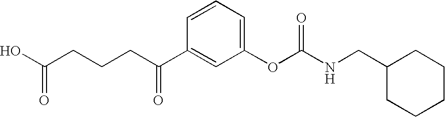

5-(3-((Cyclohexylmethyl)carbamoyl)phenyl)-5-oxopentanoic acid was prepared from cyclohexylmethylisocyanate and 5-(3-hydroxyphenyl)-5-oxopentanoic acid following the procedure outlined in Example 1.

### Example 8

### 3-(1H-Tetrazol-5-yl)phenyl cyclohexylcarbamate (Compound 10)

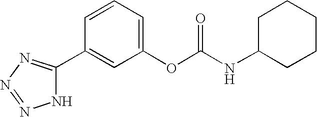

3-(1H-Tetrazol-5-yl)phenyl cyclohexylcarbamate was prepared from cyclohexyl isocyanate and 3-(1H-tetrazol-5-yl)phenol following the procedure outlined in Example 1.

### Example 9

### 3-(1H-Tetrazol-5-yl)phenyl cyclohexylmethylcarbamate (Compound 11)

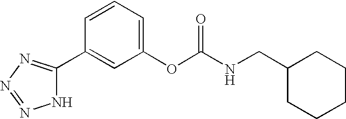

3-(1H-Tetrazol-5-yl)phenyl cyclohexylmethylcarbamate was prepared from cyclohexyl isocyanate and 3-(1H-tetrazol-5-yl)phenol following the procedure outlined in Example 1.

### Example 10

### 4-(3-(Cyclohexylcarbamoyl)phenyl)-4-oxobutanoic acid (Compound 12)

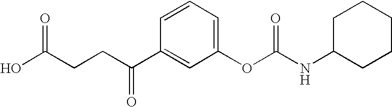

4-(3-(Cyclohexylcarbamoyl)phenyl)-4-oxobutanoic acid was prepared from cyclohexyl isocyanate and 4-(3-hydroxyphenyl)-4-oxo-butanoic acid following the procedure outlined in Example 1.

### Example 11

### 4-(3-((Cyclohexylmethyl)carbamoyl)phenyl)-4-oxobutanoic acid (Compound 13)

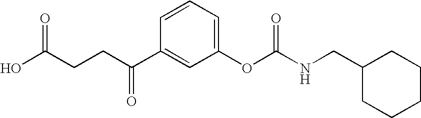

4-(3-((Cyclohexylmethyl)carbamoyl)phenyl)-4-oxobutanoic acid was prepared from cyclohexylmethylisocyanate and 4-(3-hydroxyphenyl)-4-oxo-butanoic acid following the procedure outlined in Example 1.

### Methods of Screening Compound for FAAH Inhibitory Activity

Generally, a FAAH inhibitor used in the methods described herein is identified as an inhibitor of FAAH in vitro. Preferred in vitro assays detect an increase in the level of an unaltered FAAH substrate (e.g., anandamide, OEA) or a decrease in the release of a reaction product (e.g., fatty acid amide or ethanolamine) by FAAH-mediated hydrolysis of a substrate such as AEA or OEA. The substrate may be labeled to facilitate detection of the released reaction products. High throughput assays for the presence, absence, or quantification of particular reaction products are well known to those of ordinary skill in the art. In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. Automated systems thereby allow the identification of a large number of in vitro FAAH inhibitors without undue effort.

Candidate in vivo FAAH inhibitors can be identified by their ability to increase systemic levels of one or more FAAs. Suitable FAAs include fatty acid ethanolamides with a fatty acid moiety containing 14 to 28 carbons, with 0 to 6 double bonds, such as, for example, OEA, PEA, AEA, and stearoylethanolamide (SEA). Other suitable FAAs include primary fatty acid amides with a fatty acid moiety containing 14 to 28 carbons, with 0 to 6 double bonds, such as oleamide. Biological samples from which FAA levels can be assayed include, but are not limited to, plasma, serum, blood, cerebrospinal fluid, saliva, or urine.

FAA levels in a biological sample are assayed, e.g., by liquid chromatography tandem-mass spectrometry (LC-MS/MS). Increased assay reproducibility is achieved by spiking biological samples with a known amount of an isotopically labeled FAA, which serves as an internal standard for the FAA to be assayed. The level of the FAA can also be determined using spectrophotometric techniques (e.g., a fluorometric method). Alternatively, the level of the FAA can be determined using a biological assay. In some embodiments, the level of the FAA is determined using a combination of the aforementioned techniques. Any of the foregoing assays for FAA levels can be partly or fully automated for high throughput. Details of this and other FAA assays, as well as methods for analyzing changes in FAA levels are known in the art. See, e.g., Quistad et al. (2002), Toxicology and Applied Pharmacology 179: 57-63; Quistad et al. (2001), Toxicology and Applied Pharmacology 173, 48-55; Boger et al. (2000), Proc. Natl. Acad. Sci. U.S.A. 97, 5044-49; Cravatt et al. Proc. Natl. Acad. Sci. U.S.A. 98, 9371-9376 (2001); Ramarao et al. (2005) Anal. Biochem. 343: 143-51. See also U.S. Pat. No. 6,096,784, U.S. Pat. Publication No. 2004/0127518, U.S. patent application Ser. No. 10/681,858, International Patent Publication Nos. WO 98/24396, and WO 04/033422.

### Example 12

### Compound Screening for Inhibition of FAAH Activity —FAAH LC-MS/MS Screening Assay

In one embodiment, inhibition of FAAH activity is determined using LC-MS/MS. The following are combined in a 5-mL glass tube: anandamide (5 μL of 200 ug/mL), 960 μL of 50 mM ammonium phosphate buffer (pH 7.4) containing 0.125% BSA (w/v), 10 μL of DMSO without (control) or with a FAAH inhibitor (11 g/mL), and 25 μL of human liver microsomes (31.3 μg). Prior to incubation, a 100 μL aliquot is transferred to a 96-well plate containing 0.25 mL of acetonitrile and D₄ (deuterated) anandamide (0.2 μM). Each 5-mL tube is capped and placed in a shaking water bath maintained at 37° C. for 60 minutes. After a 60 minute incubation, a second 100 μL aliquot is transferred to a 96-well plate as performed earlier. The 96-well plate is then capped, vortex mixed, and placed on an HPLC for liquid chromatography/tandem mass spectrometry (LC/MS/MS) analyses. HPLC is carried out on a Waters 2790 Alliance system (Milford, Mass.). Separation was performed on a Phenomenex C18 column (2 mm×50 mm 4μ; Torrance, Calif.) using an isocratic mobile phase of acetonitrile:water:formic acid (80:20:0.1, v/v/v) at a flow rate of 0.3 mL min⁻¹ and a column temperature of 45° C. The HPLC system was interfaced with a Micromass Ultima tandem MS (Beverly, Mass.). The samples are analyzed using an electrospray probe in the positive ionization mode with the cone voltage set at 40 V and capillary at 3.2 kV. The source and desolvation temperature settings are 130° C. and 500° C., respectively. The voltage of the CID chamber is set at −20 eV. Multiple reaction monitoring is used for the detection of anandamide as [M+H] (m/z 348>62) and D₄ anandamide (internal standard) as [M+H] (m/z 352>66). An area ratio response (anandamide area response/D₄ anandamide area response) was determined for each sample. Percent anandamide hydrolysis of each sample is determined by the following equation, [(T=60 response)−(T=0 response)/T=0 response]* 100. The percent hydrolysis normalized to control is determined by dividing the % hydrolysis of test sample by the % hydrolysis of the control sample.

For determining IC₅₀ values for candidate FAAH inhibitor compounds, the above method is used with an adjusted FAAH inhibitor concentration. In the IC₅₀ assay, the FAAH inhibitor is added at a concentration range of approximately 3 μM to 0.03 nM. The final calculation of IC₅₀ is determined by first transforming the concentrations by “X=log(X)” and then analyzing the data with a sigmoidal dose-response curve (no constraints) using GraphPad Prism version 4.00 for Windows (GraphPad Software, San Diego Calif. USA, www.graphpad.com).

### Example 13

### Compound Screening for Inhibition of FAAH Activity —FAAH Fluorescent Screening Assay

To a black 96-well plate (Nunc, cat #267342) is added 180 μL of arachidonyl 7-amino, 4-methylcoumarin amide (AAMCA, 3 μM), 20 μL of a FAAH inhibitor (0.05 μg/mL in DMSO) and 50 μL of human liver microsomes (0.25 mg/mL). The diluent for the AAMCA and human liver microsomes is fatty acid free BSA (1.4 mg/mL) in HEPES/EDTA (50 mM/1 mM) at pH 7.4. The plate is read at excitation 355 nm and emission 460 nm at T=0 on a fluorescence plate reader (SpectraMax GeminiXS, Molecular Devices) and incubated for 30 minutes at 37° C. After the 30 minute incubation, the plate is read a final time and % hydrolysis (normalized to control) was determined. The calculation for % hydrolysis is [(T=30−T=0)/T=0]*100. The percent hydrolysis normalized to control is determined by dividing the % hydrolysis of test sample by the % hydrolysis of the control sample (DMSO).

### Example 14

### Compound Screening for Inhibition of FAAH Activity—Screening for In Vivo FAAH Inhibition in Rats

Potential FAAH inhibitors are formulated for oral (p.o.), intraperitoneal (i.p.) or intravenous (i.v.) delivery to rats. Formulated compounds are administered and the animals were sacrificed at pre-determined time points post dose. At sacrifice, blood samples are collected into EDTA plasma tubes and whole brains were snap frozen in liquid nitrogen. EDTA plasma was isolated from blood samples after centrifugation. Brain and plasma samples are stored at −80° C. prior to analysis. All samples (brain and plasma) are analyzed for the concentrations of test compound (FAAH inhibitor), metabolites of the test compound and endogenous fatty acid ethanolamide levels (including anandamide, oleoylethanolamide, and palmitoylethanolamide) by LC-MS/MS. Levels of these compounds are compared across time points to determine pharmacokinetic properties of the test compounds and partial pharmacological effects of inhibiting FAAH activity (including changes of fatty acid ethanolamide levels).

In one embodiment, additional tissues and fluid samples can be collected at sacrifice. In one embodiment, FAAH activity can also be determined in fluid and tissues samples according to the methods disclosed or according to methods known in the art. In one embodiment, metabolites of the test compounds can be determined in fluid and tissue samples.

All of the compounds listed in Table 1 had IC₅₀ values less than 100 nM.

TABLE 1
Representative Compounds with IC₅₀ values less than 100 nM.

| Entry No. | Compound |
|---|---|
| 1 | KLDS-4103 (URB-597) |
| 2 | Compound 2 |
| 3 | Compound 4 |
| 4 | Compound 7 |
| 5 | Compound 11 |

### Example 15

### Determination of Pharmacokinetics

The pharmacokinetic properties of compounds provided herein were assessed in rats following oral administration as a solution. To test the oral bioavailability of compounds provided herein, a solution of the test compound was prepared for oral administration as a 10 mg/mL solutions in 80% cremephor and 20% ethanol (w/w) or as a 10 mg/mL solution of 90% PEG-400 and 10% Tween 80 (w/w). The solution of the test compound was administered to rats at a dose of 10 mg/kg via oral gavage. Table 2 shows the pK properties of representative compounds provided herein, such as Compound 2 and Compound 4, which include a moiety that is ionizable at physiological pH. As shown in Table 2, Compound 4 does not substantially cross the blood brain barrier. For comparison, the pK properties of KDS-4103 are shown in Table 2. KDS-4103 does not include a moiety that is ionizable at physiological pH.

TABLE 2
PK Properties of Alkylcarbamic aryl esters in rats
following oral administration.

| | | | Plasma | | |
|---|---|---|---|---|---|
| | | Plasma AUC | Cₘₐₓ | Brain AUC | Brain Cₘₐₓ |
| Entry | Compound | (ng * hr/mL) | (ng/mL) | (ng * hr/g) | (ng/g) |
| 1 | KDS-4103 | 6,727 | 3,161 | 9,737 | 4,584 |
| | (URB-597) | | | | |
| 2 | Compound 4 | 8,835 | 7,724 | 483 | 475 |
| 3 | Compound 2 | 1,698 | 1,247 | ND | ND |

ND = not determined

### Animal Models

Any of a variety of animal models can be used to test the compounds disclosed herein for their effectiveness in reducing inflammation and treating pain. Useful compounds can exhibit effectiveness in reducing inflammation or pain in one or more animal models.

### Animal Models for Assessing Anti-Inflammatory Activity

### Example 16

### Carrageenan-Induced Foot Pad Edema Model

The model is described, for example, by Winter et al. (1962 Proc Soc Exp Biol Med 111:544). Briefly, rats are fasted with free access to water for 17 to 19 hours before oral treatment with up to three doses of a test compound, indomethacin or celecoxib, or a control vehicle (1% methylcellulose in deionized water). One hour after the last treatment, paw edema is induced by injecting 0.05 ml of a 2% carrageenan solution into the left hindpaw. The left hindpaw volume of each rat is measured using a plethysmometer before oral treatment, at the time of carrageenan injection and at 1.5 h, 3 h, 4.5 h after the injection of carrageenan. The edema volume of each rat at each time point is expressed as the change from the volume at the time of oral treatment and the anti-inflammatory effect in treated groups is expressed as % inhibition compared to the vehicle only group 1.5 h, 3 h and 4.5 h after the carrageenan injection. The significance of the difference between in edema different groups is assessed by a one-way analysis of variance (ANOVA) followed by the non-paired Dunnett t test. In this model, hyperalgesic response and PGE₂ production can also be measured (Zhang et al. 1997 J Pharmacol and Exp Therap 283:1069).

### Example 17

### Complete Freund's Adjuvant (CFA) Induced Arthritis Model

In this model, arthritis is induced in groups of eight Lewis derived male rats weighing 160±10 g by injecting a well-ground suspension of killed Mycobacterium tuberculosis (0.3 mg in 0.1 mL of light mineral oil; Complete Freund's Adjuvant, CFA) into the subplantar region of the right hind paw on Day 1. Hind paw volumes are measured by water displacement on Days 0, 1 and 5 (right hind paw, with CFA), and on Days 0, 14 and 18 (left hind paw, without CFA); rats are weighed on Days 0 and 18. Test compounds, dissolved or suspended in 2% Tween 80, are prepared fresh daily and administered orally twice daily for 5 consecutive days (Day 1 through day 5) beginning one hour before injection of CFA. For CFA-injected vehicle control rats, the increase in paw volume on Day 5 relative to Day 1 (Acute Phase of inflammation) is generally between 0.7 and 0.9 mL; and, that on Day 18 relative to day 14 (Delayed Phase of inflammation) is generally between 0.2 and 0.4 mL. Thus, anti-inflammatory activity in this model may be denoted by values calculated during the Acute Phase as well as the Delayed Phase. Animals are also weighed on Day 0 and Day 18; CFA-injected vehicle control animals generally gain between 40 to 60 g body weight over this time period. A 30 percent or more reduction in paw volume relative to vehicle treated controls is considered of significant anti-inflammatory activity. The mean±SEM for each treatment group is determined and a Dunnett test is applied for comparison between vehicle and treated groups. Differences are considered significant at P<0.05. Polyarthritis of fore paw, tail, nose and ear can be scored visually and noted on the first day and final day, wherein positive (+) sign is for swelling response and negative (−) sign is normal. X-ray radiographies of the hindpaws can also be performed for further radiological index determination of arthritic symptoms. Hyperalgesia can also be measured in this model, allowing determination of analgesic effects of test compounds (Bertorelli et al. 1999 Brit J. Pharmacol 128:1252).

### Example 18

### Air-Pouch Model

This model is described by Masferrer et al. (1994 Proc Natl Acad Sci USA 91:3228). Briefly, male Lewis rats (175-200 g, Harlan Sprague-Dawley) are subcutaneously injected with 20 mL of sterile air into the intrascapular area of the back to create air cavities. An additional 10 mL of air is injected into the cavity every 3 days to keep the space open. Seven days after the initial air injection, 2 mL of a 1% solution of carrageenan dissolved in sterile saline is injected directly into the pouch to produce an inflammatory response. In treated and untreated animals, the volume of exudate is measured and the number of leukocytes present in the exudate is determined by Wright-Giemsa staining. In addition, PGE₂ and 6-keto-PGF_{1α} are determined in the pouch exudates from treated and untreated animals by specific ELISAs (Cayman Chemicals, Ann Arbor, Mich.).

### Animal Models for Assessing Analgesic Activity

### Example 19

### Carrageenan-Induced Thermal Hyperalgesia

This model is described by Hargreaves et al. (1988 Pain 32:77). Briefly, inflammation is induced by subplantar injection of a 2% carrageenan suspension (0.1 mL) into the right hindpaw. Three hours later, the nociceptive threshold is evaluated using a thermal nociceptive stimulation (plantar test). A light beam (44% of the maximal intensity) is focused beneath the hindpaw and the thermal nociceptive threshold is evaluated by the paw flick reaction latency (cut-off time: 30 sec). The pain threshold is measured in ipsilateral (inflamed) and in contralateral (control) hindpaws, 1 hour after the oral treatment with the test compound or a control. The results can be expressed as the nociceptive threshold in seconds (sec) for each hindpaw and the percentage of variation of the nociceptive threshold (mean±SEM) for each rat from the mean value of the vehicle group. A comparison of the nociceptive threshold between the inflamed paw and the control paw of the vehicle-treated group is performed using a Student's t test, a statistically significant difference is considered for P<0.05. Statistical significance between the treated groups and the vehicle group is determined by a Dunnett's test using the residual variance after a one-way analysis of variance (P<0.05) using SigmaStat Software.

### Example 20

### Phenylbenzoquinone-Induced Writhing Model

This model is described by Siegmund et al. (1957 Proc Soc Exp Bio Med 95:729). Briefly, one hour after oral dosing with a test compound, morphine or vehicle, 0.02% phenylbenzoquinone (PBQ) solution (12.5 mL/kg) is injected by intraperitoneal route into the mouse. The number of stretches and writhings are recorded from the 5th to the 10th minute after PBQ injection, and can also be counted between the 35th and 40th minute and between the 60th and 65th minute to provide a kinetic assessment. The results are expressed as the number of stretches and writhings (mean±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

### Example 21

### Kaolin-Induced Arthritis Model

This model is described by Hertz et al. (1980 Arzneim Forsch 30:1549). Briefly, arthritis is induced by injection of 0.1 mL of kaolin suspension into the knee joint of the right hind leg of a rat. Test compounds are administered subcutaneously after 15 minutes and again after two hours. Reference compounds can be administered orally or subcutaneously. Gait is assessed every hour from 1.5 hours to 5.5 hours after treatment and is scored as follows: normal gait (0), mild disability (1), intermittent raising of paw (2), and elevated paw (3). Results are expressed as the mean gait score (mean±SEM) calculated from individual values at each time point and the percentage of variation of the mean score calculated from the mean value of the vehicle-treated group at 4.5 hours and 5.5 hours after treatment. The statistical significance of differences between the treated groups and the vehicle-treated group is determined by a Dunnett's test using the residual variance after a one-way analysis of variance (P<0.05) at each time point.

### Example 22

### Peripheral Mononeuropathy Model

This model is described by Bennett et al. (1988 Pain 33:87) and can be used to assess anti-hyperalgesic effect of an orally administered test compound in a model of peripheral mononeuropathy. The effect of the test substance can be compared to a no treatment control or reference substance, e.g., morphine. Peripheral mononeuropathy is be induced by loose ligation of the sciatic nerve in anaesthetized male Sprague Dawley rats (pentobarbital; 45 mg/kg by intraperitoneal route). Fourteen days later, the nociceptive threshold is evaluated using a mechanical nociceptive stimulation (analgesimeter paw pressure test; Ugo Basile, Italy). The test and reference compounds and the vehicle are orally administered (10 mL/kg carried 1% methylcellulose). Increasing pressure is applied to the hindpaw of the animal until the nociceptive reaction (vocalization or paw withdrawal) is reached. The pain threshold (grams of contact pressure) is measured in ipsilateral (injured) and in contralateral (non injured) hindpaws, 60 minutes after treatment. The results are expressed as: the nociceptive threshold (mean±SEM) in grams of contact pressure for the injured paw and for the non-injured paw (vehicle-treated group) and the percentage of variation the nociceptive threshold calculated from the mean value of the vehicle-treated group. A comparison of the nociceptive threshold between the non injured paw and the injured paw of the vehicle-treated group is performed using a Student's t test. The statistical significance of the difference between the treated groups and the vehicle group is determined for the injured hindpaw by a Dunnett's test using the residual variance after a one-way analysis of variance (P<0.05) using SigmaStat Software (SigmaStat®. v. 2.0.3 (SPSS Science Software, Erkrath GmbH)).

### Example 23

### Chung Rat Model of Peripheral Neuropathy

In one embodiment, the effectiveness of a compound provided herein in alleviating neuropathic pain is demonstrated using the well-recognized Chung rat model of peripheral neuropathy. In the Chung rat model, spinal nerve partial ligation of left spinal nerves L-5 and L-6 produces a long-lasting hypersensitivity to light pressure on the affected left foot. The hypersensitivity is similar to the pain experienced by humans with the neuropathic condition of causalgia (Kim and Chung, Pain 50:355-363 (1992), which is incorporated herein by reference).

### Example 24

### Diabetic Neuropathy Paw Pressure Test

Complete protocol details can be found in Rakieten et al. (1963 Cancer Chemother Rep 29:91). Briefly, diabetes is induced by intraperitoneal injection of streptozotocin in rats. Three weeks later, the nociceptive threshold is measured using the paw pressure test to assess hyperalgesia. Test compound or controls are administered intraperitoneally 30 minutes prior to pain measurement.

### Example 25

### Acetic Acid Writhing Test

Briefly, a test compound is administered orally one hour before intraperitoneal injection of acetic acid (0.5%, 10 ml/kg) in rats. Reduction in the number of writhes by 50 percent or more (≧50) per group of animals observed during the 5 to 11 minute period after acetic acid administration, relative to a vehicle treated control group, indicates possible analgesic activity. This assay is based on that described in Inoue, K. et al. (1991 Arzneim. Forsch./Drug Res. 41: 235).

### Example 26

### Formalin Test

Complete protocol details can be found in Hunskaar et al. (1985 Neurosci. Meth. 14:69). Briefly, 30 minutes after intraperitoneal administration of a test compound or a control, 20 μL of a 5% formalin solution is injected by subplantar route into the right hindpaw of the rat. Hindpaw licking time is recorded during the early phase and the later phase after formalin injection.

### Example 27

### Tail Flick Test

Complete protocol details can be found in D'Amour and Smith (1941 J Pharmacol. Exp Ther. 72:74). Briefly, 30 minutes after intraperitoneal administration of a test compound or a control, a light beam is focused onto the tail of the rat. The nociceptive reaction latency, characterized by tail withdrawal, is recorded. The cutoff time is set to 15 seconds.

### Example 28

### Tail Immersion Test

In this test the tail of the rat is immersed into a 50-60° C. water bath. The nociceptive reaction latency, characterized by tail withdrawal, is measured (Haubrich et al. 1990 J Pharmacol Exp Ther 255:511 and Lichtman et al. 2004 Pain 109:319).

### Example 29

### Hot Plate Test

Complete protocol details can be found in Eddy et al. (1950 J Pharmacol Exp. Ther. 98:121). Briefly, 30 minutes after intraperitoneal administration of a test compound or a control, the mouse is placed on a metallic hot plate maintained at 52° C. The nociceptive reaction latency, characterized by a licking reflex of the forepaws or by a jumping off the hot plate is recorded. The cut-off time is set to 30 seconds.

### Assays for Assessing Anxiolytic Activity

Compounds provided herein that inhibit FAAH activity, and thus modulate fatty acid amide levels, may also have anxiolytic activity. Animal models to assess anxiolytic activity include:

### Example 30

### Elevated Plus Maze

The elevated plus maze consists of four maze arms that originate from a central platform, effectively forming a plus sign shape as described in van Gaalen and Steckler (2000 Behavioural Brain Research 115:95). The maze can be made of plexiglas and is generally elevated. Two of the maze arms are unwalled (open) and two are walled (closed). The two open arms are well lit and the two enclosed arms are dark (Crawley 2000 Whafs Wrong With My Mouse?: Behavioral Phenotyping of Transgenic and Knockout Mice. Wiley-Liss, New York). The test is premised on the naturalistic conflict between the tendency of an animal to explore a novel environment and the aversive properties of a brightly lit, open area (Pellow et al. 1985 J. Neuroscience Methods. 14:149).

Complete protocol details can be found in Fedorova et al. (2001 J. Pharm. Exp. Ther. 299: 332). Briefly, 15 minutes following intraperitoneal administration of test compound or control, an animal is placed individually on the central platform, facing one of the open arms opposite to the observer. The number of open and closed arm entries, and the time spent in the different compartments of the maze by the animal (central platform, open and closed arms) is scored (as described in Gaalen et al. (supra)). An arm visit is recorded when an animal moves all four paws into the arm as described in Simonin et al. (1998 EMBO J. 17: 886). Behavior is scored by an observer and/or via a video camera over a 5-minute test session. A greater amount of time spent or entries made by the animal in the open versus the closed arms is an indicator of anxiolytic activity.

### Example 31

### Elevated Zero Maze

The elevated zero maze is a modification of the elevated plus maze. The elevated zero maze consists of a plexiglas apparatus in the shape of a circle (i.e., a circular runway of 46 cm diameter and 5.5 cm runway width) with two open and two wall-enclosed sectors of equal size. It is elevated up to a meter above the ground. This apparatus is described in Simonin et al. (supra) and Crawley (supra).

Complete protocol details can be found in Kathuria et al (2003 Nature Medicine 9: 76). Briefly, 30 minutes following intraperitoneal administration of test compound or control, an animal is placed on one open sector in front of an enclosed sector. Time in a new sector is recorded as entry with all four paws. Behavior will be scored by an observer and/or via a video camera over a 5-minute test session. A greater amount of time spent or entries made by the animal in the open versus the walled sector is an indicator of anxiolytic activity.

### Example 32

### Isolation-Induced Ultrasonic Emission Test

In another animal model, the isolation-induced ultrasonic emission test, compounds provided herein are tested for their anti-anxiety effects. The isolation-induced ultrasonic emission test measures the number of stress-induced vocalizations emitted by rat pups removed from their nest (Insel, T. R. et al., Pharmacol. Biochem. Behav., 24, 1263-1267 (1986); Miczek, K. A. et al., Psychophammacology, 121, 38-56 (1995); Winslow, J. T. et al., Biol. Psychiatry, 15, 745-757 (1991); U.S. Pat. No. 6,326,156).

### Assays for Assessing Antinociception Mechanism

Compounds can be tested to determine if they influence pathways involved in nociception. The results of such assays can be used to investigate the mechanism by which a test compound mediates its antinociceptive effect.

### Example 33

### Elevation of 3α,5α-THP

3α-hydroxy-5α-pregan-20-one (3α,5α-THP or allopregnanolone) is a pregnane steroid that acts as an agonist of the inhibitory GABA_{A} receptor subtype and is known to have both anxiolytic and analgesic effects in a variety of animal systems, with supportive evidence for a similar role in humans. Thus, compounds that elevate 3α,5α-THP may have an antinociceptive effect. The level of 3α,5α-THP in the brain of animals treated with a test compound can be measured as described by VanDoren et al. (1982 J Neuroscience 20:200). Briefly, steroids are extracted from individual cerebral cortical hemispheres dissected in ice-cold saline after euthanasia. Cortices are frozen at −80° C. until use. Samples are digested in 0.3 N NaOH by sonication and extracted three times in 3 mL aliquots of 10% (v/v) ethyl acetate in heptane. The aliquots are combined and diluted with 4 mL of heptane. The extracts are applied to solid phase silica columns (Burdick & Jackson, Muskegon, Mich.), washed with pentane, and steroids of similar polarity to 3α,5α-THP are eluted off of the column by the addition of 25% (v/v) acetone in pentane. The eluant is then dried under N₂ and steroids are redissolved in 20% (v/v) isopropanol RIA buffer (0.1 M NaH₂PO₄, 0.9 M NaCl, 0.1% w/v BSA, pH 7.0). Extraction efficiency is determined in 50 μL of the redissolved extract by liquid scintillation spectroscopy and the remaining sample is used in the determination of 3α,5α-THP by radioimmunoassay. Reconstituted sample extracts (75 μL) and 3α,5α-THP standards (5-40,000 pg in 6.25% v/v ethanol, 31% v/v isopropyl alcohol in RIA buffer) are assayed in duplicate by the addition of 725 μL of RIA buffer, 100 μL of [3H] 3α,5α-THP (20,000 dpm), and 100 μL of anti-3α,5α-THP antibody. Total binding is determined in the absence of unlabeled 3α,5α-THP, and nonspecific binding is determined in the absence of antibody. The antibody-binding reaction is allowed to equilibrate for 120 min at room temperature and is terminated by cooling the mixture to 4° C. Bound 3α,5α-THP is separated from unbound 3α,5α-THP by incubation with 300 μL of cold dextran coated charcoal (DCC; 0.04% dextran, 0.4% powdered charcoal in double-distilled H₂O) for 20 min. DCC is removed by centrifugation at 2000×g for 10 min. Bound radioactivity in the supernatant is determined by liquid scintillation spectroscopy. Sample values are compared to a concurrently run 3α,5α-THP standard curve and corrected for extraction efficiency.

### Example 34

### Evaluation of Anti-Depressive Effects

In one embodiment, compounds provided herein are evaluated for anti-depressive effects in animal models. The chronic mild stress induced anhedonia model is based on the observation that chronic mild stress causes a gradual decrease in sensitivity to rewards, for example consumption of sucrose, and that this decrease is doses-dependent and reversed by chronic treatment with antidepressants. The method has previously been described by Willner, Paul, Psychopharmacology, 1997, 134, 319-329.

Another test for antidepressant activity is the forced swimming test (Nature 266, 730-732, 1977). In this test, animals are administered the compound preferably by the intraperitoneal route or by the oral route 30 or 60 minutes before the test. The animals are placed in a crystallizing dish filled with water and the time during which they remain immobile is clocked. The immobility time is then compared with that of the control group treated with distilled water. Imipramine (25 mg/kg) may be used as the positive control. The antidepressant compounds decrease the immobility time of the mice thus immersed.

Another test for antidepressant activity is the caudal suspension test on the mouse (Psychopharmacology, 85, 367-370, 1985). In this test, animals are preferably treated with a compound provided herein by the intraperitoneal route or by the oral route 30 minutes to 6 hours before the test. The animals are then suspended by the tail and their immobility time is automatically recorded by a computer system. The immobility times are then compared with those of a control group treated with vehicle. Imipramine (25 mg/kg) may be used as the positive control. Antidepressant compounds decrease the immobility time of the mice.

Antidepressant effects of the compounds provided herein can be tested in the DRL-72 TEST. This test, carried out according to the protocol of Andrews et al “Effects of imipramine and mirtazapine on operant performance in rats” Drug Development Research 32, 5 8-66 (1994), gives an indication of antidepressant-like activity. The effects of the compounds provided herein also may be examined in serotonin disorders and bipolar disorders, such as described in U.S. Pat. Nos. 6,403,573 and 5,952,315, incorporated herein by reference.

### Example 35

### Evaluation of Anticovulsant Effects

In another embodiment, compounds provided herein are examined for anticonvulsant activity in animal models, as described in U.S. Pat. Nos. 6,309,406 and 6,326,156.

### Example 36

### Effects of Compounds on Appetite Behaviour

In one embodiment, compounds provided herein are administered to a rat in order to measure the effect on appetite behavior. The effect of the administered compound is assessed by examining the intake of a sucrose solution by the rat. This method is taught in W. C. Lynch et al., Physiol. Behav., 1993, 54, 877-880. Male Sprague-Dawley rats weighing about 190 g to about 210 g are under a normal light cycle (from 7 am to 7 pm) and receive water and food ad libitum. For 6 days, between 11 am and 3 pm, the food and the water bottles are withdrawn and the rats are given a 5% sucrose solution to drink. Rats drinking less than 3 g of sucrose solution are eliminated. On the seventh day the test is carried out according to the following procedure: 9 am: withdrawal of food, 10 am: administration of either a compound provided herein or vehicle to the test animals; 11 am=T0: introduction of bottles containing a weighed sucrose solution; T0+1 hour, T0+2 hours, T0+3 hours, T0+4 hours: measurement of the sucrose consumption by weighing of the bottles. Followed by comparison of the experimental (administered a compound provided herein) and control groups' intake of the sucrose solution. Animals can be, for example, obese or normal guinea pigs, rats, mice, or rabbits. Suitable rats include, for example, Zucker rats. Suitable mice include, for example, normal mice, ALS/LtJ, C3.5W-H-2b/SnJ, (NON/LtJ×NZO/HlJ)Fl, NZO/HlJ, ALR/LtJ, NON/LtJ, KK.Cg-AALR/LtJ, NON/LtJ, KK.CgAy/J, B6.HRS(BKS)-Cpefat/+, B6.129P2-GcktmlEfr, B6.V-Lepob, BKS.Cg-m+1+Leprdb, and C57BL/6J with Diet Induced Obesity.

In another test, the effect of a compound of the invention on the consumption of an alcohol solution can be shown in mice. For instance, male C57BL6 mice are isolated on the day of their arrival in an animal housing under a reverse cycle (night from 10 am to 10 pm) with 2 bottles filled with water. After 1 week, one of the bottles of water is replaced with a bottle filled with a 10% alcohol solution for 6 hours of the test. Each day, 30 minutes before the bottle of alcohol is introduced, the mice are treated with a compound of the invention. The amounts of alcohol and water consumed are measured after 6 hours. The test is repeated for 4 days. The results for an experimental and a control or vehicle are compared.

### Example 37

### Reduction of Body Weight, Body Fat, and Liver Steatosis

The effects of inhibiting FAAH activity on body weight, body fat, triglyceride levels, cholesterol levels can be determined in APOE*3-Leiden transgenic (E3L) mice, an animal model of hyperlipidemia. E3L mice express a mutated variant of human apoE, apoE*3-Leiden, that has impaired binding of apoE to the LDL receptor. Consequently, E3L mice exhibit a decreased clearance rate of apoB-containing lipoproteins and elevated serum lipid levels. See van Vlijmen et al. (1994), J. Clin Invest., 93:1403-1410. Upon high fat and cholesterol feeding, these mice develop various stages of atherosclerotic lesions depending on plasma total cholesterol levels and resembling those found in humans. See Groot et al. (1996), Arterioscler. Thromb. Vasc. Biol., 16:926-933; Verschuren et al. (2005), Arterioscler. Thromb. Vasc Biol., 25:161-167; and Lutgens et al. (1999), Circulation; 99(2):276-283). Thus, the E3L mouse is a suitable model for the investigation of the efficacy of anti-atherosclerotic drugs.

E3L mice are fed a high cholesterol (1% w/w) diet (HC diet) for a period of four weeks. Animals are then matched based on their plasma cholesterol levels, and are divided into five groups, each of which was maintained on an HC diet. Every day for the remainder of the study (four weeks), a “control” group receives food with no additives, a “fenofibrate” group receives food containing fenofibrate (0.04% w/w), an “oral vehicle” group receives an oral suspension of vehicle, an “oral OEA” group receives an oral suspension of OEA at a dose of 500 mg/kg, and an “oral Carbamate” group receives an oral suspension of a compound provided herein at a dose of 10 mg/kg.

Blood samples are collected at days 0, 14, and 28 of the treatment period. At the end of the treatment period, animals are sacrificed, and various tissues and organs are analyzed.

### Example 38

### Cannabinoid Receptor Binding

Compounds may exert an antinociceptive effect via binding to either or both of the cannabinoid receptors CB₁ and CB₂. CB₁ is expressed in the brain (Matsuda et al. 1990 Nature 346:561), and CB₂ is expressed by macrophages and in the spleen (Munro et al. 1993 Nature 365:61). Both of these receptors have been implicated in mediating analgesic effects through binding of agonists (see, for example, Clayton et al. 2002 Pain 96:253). Thus, test compounds can be assayed to determine whether they bind to one or both human cannabinoid receptors. An assay for CB₁ binding is described by Matsuda et al. (supra). This assay employs recombinant cells expressing CB₁. Binding to CB₂ can be determined in the same manner using recombinant cells expressing CB₂. Briefly, to measure the ability of a test compound to bind to CB₁, the binding of a labelled CB₁, ligand, e.g., [³H]WIN 55212-2 (2 nM for CB₁ and 0.8 nM for CB₂) to membranes isolated from HEK-293 cells expressing recombinant CB₁ is measured in the presence and absence of a compound. Non-specific binding is separately determined in the presence of several-fold excess of unlabelled WIN 55212-2 (5 μM for CB₁ and 10 uM for CB₂). The specific ligand binding to the receptors is defined as the difference between the total binding and the non-specific binding determined in the presence of an excess of unlabelled WIN 55212-2. The IC₅₀ values and Hill coefficients (n_{H}) are determined by non-linear regression analysis of the competition curves using Hill equation curve fitting. The inhibition constants (Kᵢ) are calculated from the Cheng Prusoff equation (Kᵢ=IC₅₀/(1+(L/K_{D})), where L=concentration of radioligand in the assay, and K_{D}=affinity of the radioligand for the receptor).

### Example 39

### Pharmaceutical Compositions

### Example 39a

### Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example 39b

### Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example 39c

### Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 39d

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 39e

### Rectal Gel Composition

To prepare a pharmaceutical composition for rectal delivery, 100 mg of a compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 39f

### Topical Gel Composition

To prepare a pharmaceutical topical gel composition, 100 mg of a compound described herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 39g

### Ophthalmic Solution Composition

To prepare a pharmaceutical ophthalmic solution composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

The examples and embodiments described herein are for illustrative purposes only and various modifications or changes suggested to persons skilled in the art are to be included within the spirit and purview of this application and scope of the appended claims. All publications, patents, and patent applications cited herein are hereby incorporated by reference for all purposes.

What is claimed is:

1. A compound of Formula (II): 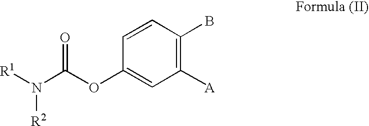
wherein:
R¹ is an optionally substituted group selected from among C₃-C₉ cycloalkyl and —C₁-C₄alkyl(C₃-C₉cycloalkyl), wherein any carbon of the R¹ carbocyclic ring can be optionally substituted by both Y and Z;
R² is H or an optionally substituted alkyl;
Y and Z are independently selected from among halogen or methyl; or
Y and Z taken together form an oxo (═O);
one of A or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —S(O)₂NH—(CH₂)ₙ—, —C(═NR¹⁰)N(R⁹)—(CH₂)ₙ—, and —NR⁹C(═NR¹⁰)N(R⁹)—(CH₂)ₙ—;
G is tetrazolyl, —NHS(═O)₂R⁸, —S(═O)₂NHR⁹, —OH, —SH, —N(R⁹)₂, —C(O)NHC(O)R⁸, —C(O)NHS(═O)₂R⁸, —S(═O)₂NHC(O)R¹, —S(═O)₂NHC(O)NHR⁸, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, a carboxylic acid bioisostere, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 0, 1, or 2; and
pharmaceutically acceptable salts, pharmaceutically acceptable N-oxides, pharmaceutically active metabolites, pharmaceutically acceptable prodrugs, or pharmaceutically acceptable solvates thereof.

2. The compound of claim 1, wherein one of A or B is -L-G and the other is H.

3. The compound of claim 1, wherein A is -L-G.

4. The compound of claim 1, wherein B is -L-G.

5. The compound of claim 1, wherein R² is H.

6. The compound of claim 1, wherein:
L is a bond, or an optionally substituted group selected from among C₁-C₆ alkylene, C₁-C₆ ketoalkylene, —C(O)NR⁹—(CH₂)ₙ—, —NR⁹—C(O)—(CH₂)ₙ—, —S(O)₂—(CH₂)ₙ—, and —S(O)₂NH—(CH₂)ₙ—.

7. The compound of claim 6, wherein L is a bond.

8. The compound of claim 1, wherein if L is not a bond, L taken together with A or B can form a carbocyclic ring.

9. The compound of claim 1, wherein:
G is —N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, NR⁹C(═CHR¹⁰)N(R⁹)₂, —C(O)NR⁹C(═NR¹⁰)N(R⁹)₂, or —C(O)NR⁹C(═CHR¹⁰)N(R⁹)₂.

10. The compound of claim 1, wherein:
each R¹⁰ is H.

11. The compound of claim 1, wherein:
G is tetrazolyl, —NHS(═O)₂R⁸, —S(═O)₂NHR⁹, —OH, —SH, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)ₙOH; —OS(O)_{y}OH; or —NR⁹S(O)_{y}OH.

12. The compound of claim 1, wherein:
G is tetrazolyl, —C(O)NHC(O)R⁸, —C(O)NHS(═O)₂R⁸, —S(═O)₂NHC(O)R⁸, —S(═O)₂NHC(O)NHR⁸, —CO₂H, a carboxylic acid bioisostere, —(OP(═O)OH)ₓOH, —OP(═O)OR⁸OH, —OP(═O)R⁸OH, —P(═O)OR⁸OH; —P(═O)R⁸OH, or —S(O)_{y}OH.

13. The compound of claim 1, wherein:
G is tetrazolyl, —C(O)NHC(O)R⁸, —C(O)NHS(═O)₂R⁸, —S(═O)₂NHC(O)R⁸, —S(═O)₂NHC(O)NHR⁸, —CO₂H, carboxylic acid bioisosteres, —(OP(═O)OH)ₓOH, or —S(O)_{y}OH;
x is 1; and
y is 1 or 2.

14. The compound of claim 1, wherein:
R¹ is selected from among cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, cycloheptylmethyl and cyclooctylmethyl.

15. The compound according to claim 15, wherein:
R¹ is selected from among cyclohexyl and cyclohexylmethyl.

16. A compound of claim 1, wherein the compound is selected from among:
3-(cyclohexylcarbamoyloxy)benzoic acid (Compound 1);
3-(cyclohexylmethylcarbamoyloxy)benzoic acid (Compound 2);
4-(cyclohexylcarbamoyloxy)benzoic acid (Compound 3);
4-(cyclohexylmethylcarbamoyloxy)benzoic acid (Compound 4);
2-(3-((cyclohexylmethyl)carbamoyl)phenyl)acetic acid (Compound 7);
5-(3-(cyclohexylcarbamoyl)phenyl)-5-oxopentanoic acid (Compound 8);
5-(3-((cyclohexylmethyl)carbamoyl)phenyl)-5-oxopentanoic acid (Compound 9);
3-(1H-tetrazol-5-yl)phenyl cyclohexylcarbamate (Compound 10);
3-(1H-tetrazol-5-yl)phenyl cyclohexylmethylcarbamate (Compound 11);
4-(3-(cyclohexylcarbamoyl)phenyl)-4-oxobutanoic acid (Compound 12); and
4-(3-((cyclohexylmethyl)carbamoyl)phenyl)-4-oxobutanoic acid (Compound 13).

17. A pharmaceutical composition comprising a compound, pharmaceutically acceptable salt, pharmaceutically acceptable N-oxide, pharmaceutically active metabolite, pharmaceutically acceptable prodrug, or pharmaceutically acceptable solvate of claim 1 and a pharmaceutically acceptable diluent, excipient or binder.

18. A method of inhibiting the activity of fatty acid amide hydrolase or of treating a disease, disorder, or condition, which would benefit from inhibition of fatty acid amide hydrolase activity in a patient comprising administering to the patient a therapeutically effective amount of a compound, pharmaceutically acceptable salt, pharmaceutically acceptable N-oxide, pharmaceutically active metabolite, pharmaceutically acceptable prodrug, or pharmaceutically acceptable solvate of claim 1.

19. The method of claim 23, wherein the disease, disorder or condition is selected from among acute or chronic pain, eating disorders, cardiovascular diseases, metabolic diseases, disorders or conditions, renal ischemia, cancers, disorders of the immune system, allergic diseases, parasitic, viral or bacterial infectious diseases, inflammatory diseases, osteoporosis, ocular conditions, pulmonary conditions, gastrointestinal diseases and urinary incontinence.

20. The method of claim 19, wherein said compound is an irreversible inhibitor of fatty acid amide hydrolase.

21. The method of claim 21, wherein said compound does not substantially cross the blood-brain barrier.

22. Use of a compound of claim 1 for inhibiting the activity of fatty acid amide hydrolase activity or for the treatment of a disease, disorder, or condition, that would benefit from inhibition of fatty acid amide hydrolase activity.

23. Use of a compound of claim 1 for the formulation of a medicament for the inhibition of fatty acid amide hydrolase (FAAH).

24. An article of manufacture, comprising packaging material, a compound of claim 1, which is effective for inhibiting the activity of fatty acid amide hydrolase (FAAH), within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for inhibiting the activity of fatty acid amide hydrolase (FAAH).

25. A compound having the structure of Formula (I): 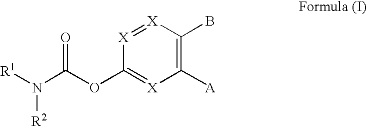
wherein:
R¹ is an optionally substituted group selected from among C₁-C₆ alkyl, C₃-C₉ cycloalkyl and —C₁-C₄alkyl(C₃-C₉cycloalkyl);
R² is H or an optionally substituted alkyl;
if R¹ is an optionally substituted C₃-C₉ cycloalkyl or an optionally substituted —C₁-C₄alkyl(C₃-C₉cycloalkyl), then any carbon of the R¹ carbocyclic ring can be optionally substituted by both Y and Z;
Y and Z are independently selected from among halogen or methyl; or
Y and Z taken together form an oxo (═O);
one of A or B is -L-G and the other is selected from among H and an optionally substituted C₁-C₆ alkyl; or
A and B are each independently selected from among H and an optionally substituted C₁-C₆ alkyl provided that at least one X is N;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ, —S(O)₂—(CH₂)ₙ—, —S(O)₂NH—(CH₂)ₙ—, —C(═NR¹⁰)N(R⁹)—(CH₂)ₙ—, and —NR⁹C(═NR¹⁰)N(R⁹)—(CH₂)ₙ—;
G is tetrazolyl, —NHS(═O)₂R⁸, —S(═O)₂NHR⁹, —OH, —SH, —N(R⁹)₂, —C(O)NHC(O)R⁸, —C(O)NHS(═O)₂R⁸, —S(═O)₂NHC(O)R⁸, —S(═O)₂NHC(O)NHR⁸, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R)₂, —C(O)NR⁹C(═CHR¹⁰)N(R⁹)₂, —CO₂H, a carboxylic acid bioisostere, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; or —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 0, 1, or 2; and
pharmaceutically acceptable salts, pharmaceutically acceptable N-oxides, pharmaceutically active metabolites, pharmaceutically acceptable prodrugs, or pharmaceutically acceptable solvates thereof.
